SEQUENCE LISTING (99 sequences)

SEQ ID NO: 1 - DNA, 1139 bp, Rattus sp.
  CDS (33)..(494)
caggtctagagttggaatcgaagcctcttaaaatggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaataactggcttccagggtggcggtggtggcagcagtgatccatgagcctacagaggcccctcccccagctctggctgggcccttggctggactcctatccaatttatttgacgttttattttggttttcctcaccccttcaaactgtcggggagaccctgcccttcacctagctcccttggccaggcatgagggagccatggccttggtgaagctacctgcctcttctctcgcagccctgatgggggaaagggagtgggtactgcctgtggtttaggttcccctctccctttttctttttaattcaatttggaatcagaaagctgtggattctggcaaatggtcttgtgtcctttatcccactcaaacccatctggtcccctgttctccatagtccttcacccccaagcaccactgacagactggggaccagcccccttccctgcctgtgtctcttcccaaacccctctataggggtgacaagaagaggagggggggaggggacacgatccctcctcaggcatctgggaaggccttgcccccatgggctttaccctttcctgtgggctttctccctgacacatttgttaaaaatcaaacctgaataaaactacaagtttaatatgaaaaaaaaaaaaaaaaaaaaa

SEQ ID NO: 2 - protein, 154 aa, Rattus sp.
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 3 - DNA, 462 bp, Homo sapiens
atggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaa

SEQ ID NO: 4 - DNA, 460 bp, Homo sapiens
atggcagacgaaattgatttcactactggagatgccggggcttccagcacttaccctatgcagtgctcggccttgcgcaaaaacggcttcgtggtgctcaaaggacgaccatgcaaaatagtggagatgtcaacttccaaaactggaaagcatggtcatgccaaggttcaccttgttggaattgatattttcacgggcaaaaaatatgaagatatttgtccttctactcacaacatggatgttccaaatattaagagaaatgattatcaactgatatgcattcaagatggttacctttccctgctgacagaaactggtgaagttcgtgaggatcttaaactgccagaaggtgaactaggcaaagaaatagagggaaaatacaatgcaggtgaagatgtacaggtgtctgtcatgtgtgcaatgagtgaagaatatgctgtagccataaaaccctgcaaat

SEQ ID NO: 5 - DNA, 462 bp, Rattus sp.
atggcagatgatttggacttcgagacaggagatgcaggggcctcagccaccttcccaatgcagtgctcagcattacgtaagaatggttttgtggtgctcaaaggccggccatgtaagatcgtcgagatgtctacttcgaagactggcaagcatggccatgccaaggtccatctggttggcattgacatttttactgggaagaaatatgaagatatctgcccgtcgactcataatatggatgtccccaacatcaaacggaatgacttccagctgattggcatccaggatgggtacctatccctgctccaggacagtggggaggtacgagaggaccttcgtctgcctgaaggagaccttggcaaggagattgagcagaagtatgactgtggagaagagatcctgatcacagtgctgtctgccatgacagaggaggcagctgttgcaatcaaggccatggcaaaa

SEQ ID NO: 6 - DNA, 606 bp, Rattus sp.
  CDS (1)..(453)
gctgtgtattattgggcccataagaaccacatacctgtgctgagtcctgcactcacagacggctcactgggtgacatgatctttttccattcctataaaaacccaggcttggtcctggacatcgttgaagacctgcggctcatcaacatgcaggccattttcgccaagcgcactgggatgatcatcctgggtggaggcgtggtcaagcaccacatcgccaatgctaacctcatgcggaatggagctgactacgctgtttatatcaacacagcccaggagtttgatggctcagactcaggagcccggccagatgaggctgtctcctggggcaagatccggatggatgcacagccagtaaaggtctatgctgatgcatctctggttttccccttgctggtggctgagacattcgcccaaaaggcagatgccttcagagctgagaagaatgaggactgagcagatgggtaaagacggaggcttctgccacacctttatttattatttgcataccaacccctcctgggccctctccttggtcagcagcatcttgagaataaatggcctttttgttggtttctgtaaaaaaaggactttaaaaaaaaaaaa

SEQ ID NO: 7 - protein, 151 aa, Rattus sp.
AVYYWAHKNHIPVLSPALTDGSLGDMIFFHSYKNPGLVLDIVEDLRLINMQAIFAKRTGMIILGGGVVKHHIANANLMRNGADYAVYINTAQEFDGSDSGARPDEAVSWGKIRMDAQPVKVYADASLVFPLLVAETFAQKADAFRAEKNED

SEQ ID NO: 8 - DNA, 453 bp, Homo sapiens
tccgtgtattactgggcccagaagaaccacatccctgtgtttagtcccgcacttacagacggctcgctgggcgacatgatcttcttccattcctacaagaacccgggcctggtcctggacatcgttgaggacctgaggctcatcaacacacaggccatctttgccaagtgcactgggatgatcattctgggcgggggcgtggtcaagcaccacattgccaatgccaacctcatgcggaacggggccgactacgctgtttacatcaacacagcccaggagtttgatggctctgactcaggtgcccgaccagacgaggctgtctcctggggcaagatccgggtggatgcacagcccgtcaaggtctatgctgacgcctccctggtcttccccctgcttgtggctgaaacctttgcccagaagatggatgccttcatgcatgagaagaacgaggac

SEQ ID NO: 9 - DNA, 20 bp, Artificial Sequence
  Description of Artificial Sequence Synthetic Primer
  modified_base (12): a, t, c or g
tcsaarachggnaagcaygg

SEQ ID NO: 10 - DNA, 42 bp, Artificial Sequence
  Description of Artificial Sequence Synthetic Primer
gcgaagcttccatggctcgagttttttttttttttttttttt

SEQ ID NO: 11 - DNA, 972 bp, Rattus sp.
  CDS (1)..(327)
tcgaagaccggtaagcacggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaataactggcttccagggtggcggtggtggcagcagtgatccatgagcctacagaggcccctcccccagctctggctgggcccttggctggactcctatccaatttatttgacgttttattttggttttcctcaccccttcaaactgtcggggagaccctgcccttcacctagctcccttggccaggcatgagggagccatggccttggtgaagctacctgcctcttctctcgcagccctgatgggggaaagggagtgggtactgcctgtggtttaggttcccctctccctttttctttttaattcaatttggaatcagaaagctgtggattctggcaaatggtcttgtgtcctttatcccactcaaacccatctggtcccctgttctccatagtccttcacccccaagcaccactgacagactggggaccagcccccttccctgcctgtgtctcttcccaaacccctctataggggtgacaagaagaggagggggggaggggacacgatccctcctcaggcatctgggaaggccttgcccccatgggctttaccctttcctgtgggctttctccctgacacatttgttaaaaatcaaacctgaataaaactacaagtttaatatgaaaaaaaaaaaaaaaaaaaaa

SEQ ID NO: 12 - protein, 109 aa, Rattus sp.
SKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKAMAK

SEQ ID NO: 13 - DNA, 24 bp, Artificial Sequence
  Description of Artificial Sequence Synthetic Primer
caggtctagagttggaatcgaagc

SEQ ID NO: 14 - DNA, 30 bp, Artificial Sequence
  Description of Artificial Sequence Synthetic Primer
atatctcgagccttgattgcaacagctgcc

SEQ ID NO: 15 - DNA, 489 bp, Rattus sp.
  CDS (33)..(485)
caggtctagagttggaatcgaagcctcttaaaatggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggctcgag

SEQ ID NO: 16 - protein, 151 aa, Rattus sp.
MADDLDFETGDAGASATFPMQCSALRKNGFVVLKGRPCKIVEMSTSKTGKHGHAKVHLVGIDIFTGKKYEDICPSTHNMDVPNIKRNDFQLIGIQDGYLSLLQDSGEVREDLRLPEGDLGKEIEQKYDCGEEILITVLSAMTEEAAVAIKA

SEQ ID NO: 17 - DNA, 20 bp, Artificial Sequence
  Description of Artificial Sequence Synthetic Primer
gtctgtgtattattgggccc

SEQ ID NO: 18 - DNA, 42 bp, Artificial Sequence; identical to SEQ ID NO: 10
  Description of Artificial Sequence Synthetic Primer

SEQ ID NO: 19 - DNA, 1299 bp, Homo sapiens
ggcacgagggcggcggcggcggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtcttttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaa

SEQ ID NO: 20 - DNA, 462 bp, Rattus sp.
atggcagatgatttggacttcgagacaggagatgcaggggcctcagccaccttcccaatgcagtgctcagcattacgtaagaatggttttgtggtgctcaagggccggccatgtaagatcgtcgagatgtctacttcgaagactggcaagcatggccatgccaaggtccatctggttggtattgatatttttactgggaagaaatatgaagatatctgcccgtcgactcataacatggatgtccccaacatcaaaaggaatgatttccagctgattggcatccaggatgggtacctatccctgctccaggacagtggggaggtacgagaggaccttcgtctgcctgagggagaccttggcaaggagattgagcagaagtatgactgtggagaagagatcctgatcacagtgctgtccgccatgacagaggaggcagctgttgcaatcaaggccatggcaaaa

SEQ ID NO: 21 - protein, 154 aa, Homo sapiens; identical to SEQ ID NO: 2

SEQ ID NO: 22 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGYLSLLTETGEVREDLKLPEGELGKEIEGKYNAGEDVQVSVMCAMSEEYAVAIKPCK

SEQ ID NO: 23 - protein, 154 aa, Mus musculus; identical to SEQ ID NO: 2

SEQ ID NO: 24 - protein, 153 aa, Homo sapiens
MADEIDFTTGDAGASSTYPMQCSALRKNGFVVLKGRPCKIVEMSTSKTGKHGHAKVHLVGIDIFTGKKYEDICPSTHNMDVPNIKRNDYQLICIQDGCLSLLTETGEVREDLKLPEGELGKEIEGKYNAGEDVQVSVMCAMSEEYAVAIKPCK

SEQ ID NO: 25 - DNA, 20 bp, Artificial Sequence
  Description of Artificial Sequence Synthetic oligonucleotide
gacttggacttcgagacagg

SEQ ID NO: 26 - DNA, 19 bp, Artificial Sequence
  Description of Artificial Sequence Synthetic oligonucleotide
gcacggccacgccaaggtc

SEQ ID NO: 27 - DNA, 20 bp, Artificial Sequence
  Description of Artificial Sequence Synthetic oligonucleotide
ggacagcggggaggtacgag

SEQ ID NO: 28 - protein, 153 aa, Artificial Sequence; identical to SEQ ID NO: 24
  Description of Artificial Sequence Synthetic consensus sequence

SEQ ID NO: 29 - DNA, 1309 bp, Homo sapiens
ggcacgagggtagaggcggcggcggcggcggcagcgggctcggaggcagcggttgggctcgcggcgagcggacggggtcgagtcagtgcgttcgcgcgagttggaatcgaagcctcttaaaatggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataactggctcccaggatggcggtggtggcagcagtgatcctctgaacctgcagaggccccctccccgagcctggcctggctctggcccggtcctaagctggactcctcctacacaatttatttgacgttttattttggttttccccaccccctcaatctgtcggggagcccctgcccttcacctagctcccttggccaggagcgagcgaagctgtggccttggtgaagctgccctcctcttctcccctcacactacagccctggtgggggagaagggggtgggtgctgcttgtggtttagtctttttttttttttttttttttttttaaattcaatctggaatcagaaagcggtggattctggcaaatggtccttgtgccctccccactcatccctggtctggtcccctgttgcccatagccctttaccctgagcaccaccccaacagactggggaccagccccctcgcctgcctgtgtctctccccaaacccctttagatggggagggaagaggaggagaggggaggggacctgccccctcctcaggcatctgggagggccctgcccccatgggctttacccttccctgcgggctctctccccgacacatttgttaaaatcaaacctgaataaaactacaagtttaatatgaaaaaaaaaaaaaaaaaaaaaaaaaaaaaaaaaaaaaaaaaaaaaaaaaaaaaaaaaaaaaaaaaaaaaaaaaa

SEQ ID NO: 30 - DNA, 23 bp, Homo sapiens
aaaggaatgacttccagctgatt

SEQ ID NO: 31 - DNA, 23 bp, Homo sapiens
aagatcgtcgagatgtctacttc

SEQ ID NO: 32 - DNA, 23 bp, Homo sapiens
aaggtccatctggttggtattga

SEQ ID NO: 33 - DNA, 23 bp, Homo sapiens
aagctggactcctcctacacaat

SEQ ID NO: 34 - DNA, 23 bp, Homo sapiens
aaagtcgaccttcagtaaggatt

SEQ ID NO: 35 - DNA, 20 bp, Homo sapiens
cctgtctcgaagtccaagtc

SEQ ID NO: 36 - DNA, 20 bp, Homo sapiens
gacttggacttcgagacagg

SEQ ID NO: 37 - DNA, 20 bp, Homo sapiens
ggaccttggcgtggccgtgc

SEQ ID NO: 38 - DNA, 20 bp, Homo sapiens
gcacggccacgccaaggtcc

SEQ ID NO: 39 - DNA, 20 bp, Homo sapiens
ctcgtacctccccgctctcc

SEQ ID NO: 40 - DNA, 19 bp, Homo sapiens
ggacagcggggaggtacga

SEQ ID NO: 41 - DNA, 465 bp, Homo sapiens
atggcagatgacttggacttcgagacaggagatgcaggggcctcagccaccttcccaatgcagtgctcagcattacgtaagaatggctttgtggtgctcaaaggccggccatgtaagatcgtcgagatgtctacttcgaagactggcaagcacggccacgccaaggtccatctggttggtattgacatctttactgggaagaaatatgaagatatctgcccgtcaactcataatatggatgtccccaacatcaaaaggaatgacttccagctgattggcatccaggatgggtacctatcactgctccaggacagcggggaggtacgagaggaccttcgtctccctgagggagaccttggcaaggagattgagcagaagtacgactgtggagaagagatcctgatcacggtgctgtctgccatgacagaggaggcagctgttgcaatcaaggccatggcaaaataa

SEQ ID NO: 42 - DNA, 462 bp, Homo sapiens
atggcagacgaaattgatttcactactggagatgccggggcttccagcacttaccctatgcagtgctcggccttgcgcaaaaacggcttcgtggtgctcaaaggacgaccatgcaaaatagtggagatgtcaacttccaaaactggaaagcatggtcatgccaaggttcaccttgttggaattgatattttcacgggcaaaaaatatgaagatatttgtccttctactcacaacatggatgttccaaatattaagagaaatgattatcaactgatatgcattcaagatggttacctttccctgctgacagaaactggtgaagttcgtgaggatcttaaactgccagaaggtgaactaggcaaagaaatagagggaaaatacaatgcaggtgaagatgtacaggtgtctgtcatgtgtgcaatgagtgaagaatatgctgtagccataaaaccctgcaaataa

SEQ ID NO: 43 - protein, 154 aa, Homo sapiens [sequence omitted]

SEQ ID NO: 44 - DNA, 21 bp, Artificial Sequence
  Description of Artificial Sequence Synthetic oligonucleotide
aaaggaatgacttccagctga

SEQ ID NO: 45 - DNA, 23 bp, Artificial Sequence
  Description of Combined DNA/RNA Molecule Synthetic oligonucleotide
  Description of Artificial Sequence Synthetic oligonucleotide
aaaggaaugacuuccagcugatt

SEQ ID NO: 46 - DNA, 23 bp, Artificial Sequence
  Description of Combined DNA/RNA Molecule Synthetic oligonucleotide
  Description of Artificial Sequence Synthetic oligonucleotide
ucagcuggaagucauuccuuutt

SEQ ID NO: 47 - DNA, 21 bp, Artificial Sequence
  Description of Artificial Sequence Synthetic oligonucleotide
aagatcgtcgagatgtctact

SEQ ID NO: 48 - DNA, 23 bp, Artificial Sequence
  Description of Combined DNA/RNA Molecule Synthetic oligonucleotide
  Description of Artificial Sequence Synthetic oligonucleotide
aagaucgucgagaugucuacutt

SEQ ID NO: 49 - DNA, 23 bp, Artificial Sequence
  Description of Combined DNA/RNA Molecule Synthetic oligonucleotide
  Description of Artificial Sequence Synthetic oligonucleotide
aguagacaucucgacgaucuutt

SEQ ID NO: 50 - DNA, 21 bp, Artificial Sequence
  Description of Artificial Sequence Synthetic oligonucleotide
aaggtccatctggttggtatt

SEQ ID NO: 51 - DNA, 23 bp, Artificial Sequence
  Description of Combined DNA/RNA Molecule Synthetic oligonucleotide
  Description of Artificial Sequence Synthetic oligonucleotide
aagguccaucugguugguauutt

SEQ ID NO: 52 - DNA, 23 bp, Artificial Sequence
  Description of Combined DNA/RNA Molecule Synthetic oligonucleotide
  Description of Artificial Sequence Synthetic oligonucleotide
aauaccaaccagauggaccuutt

SEQ ID NO: 53 - DNA, 21 bp, Artificial Sequence
  Description of Artificial Sequence Synthetic oligonucleotide
aagctggactcctcctacaca

SEQ ID NO: 54 - DNA, 23 bp, Artificial Sequence
  Description of Combined DNA/RNA Molecule Synthetic oligonucleotide
  Description of Artificial Sequence Synthetic oligonucleotide
aagcuggacuccuccuacacatt

SEQ ID NO: 55 - DNA, 23 bp, Artificial Sequence
  Description of Combined DNA/RNA Molecule Synthetic oligonucleotide
  Description of Artificial Sequence Synthetic oligonucleotide
uguguaggaggaguccagcuutt

SEQ ID NO: 56 - DNA, 21 bp, Artificial Sequence
  Description of Artificial Sequence Synthetic oligonucleotide
aaagtcgaccttcagtaagga

SEQ ID NO: 57 - DNA, 23 bp, Artificial Sequence
  Description of Combined DNA/RNA Molecule Synthetic oligonucleotide
  Description of Artificial Sequence Synthetic oligonucleotide
aaagucgaccuucaguaaggatt

SEQ ID NO: 58 - DNA, 23 bp, Artificial Sequence
  Description of Combined DNA/RNA Molecule Synthetic oligonucleotide
  Description of Artificial Sequence Synthetic oligonucleotide
uccuuacugaaggucgacuuutt

SEQ ID NO: 59 - DNA, 26 bp, Artificial Sequence
  Description of Artificial Sequence Synthetic primer
gccaagcttaatggcagatgatttgg

SEQ ID NO: 60 - DNA, 25 bp, Artificial Sequence
  Description of Artificial Sequence Synthetic primer
ctgaattccagttattttgccatgg

SEQ ID NO: 61 - DNA, 27 bp, Artificial Sequence
  Description of Artificial Sequence Synthetic primer
aatgaattccgccatgacagaggaggc

SEQ ID NO: 62 - DNA, 42 bp, Artificial Sequence; identical to SEQ ID NO: 10
  Description of Artificial Sequence Synthetic primer

SEQ ID NO: 63 - DNA, 20 bp, Artificial Sequence
  Description of Artificial Sequence Synthetic oligonucleotide
cctgtctcgaagtccaagtc

SEQ ID NO: 64 - DNA, 20 bp, Artificial Sequence
  Description of Artificial Sequence Synthetic oligonucleotide
ggaccttggcgtggccgtgc

SEQ ID NO: 65 - DNA, 20 bp, Artificial Sequence
  Description of Artificial Sequence Synthetic oligonucleotide
ctcgtacctccccgctctcc

SEQ ID NO: 66 - DNA, 20 bp, Artificial Sequence
  Description of Artificial Sequence Synthetic oligonucleotide
cgtaccggtacggttccagg

SEQ ID NO: 67 - DNA, 20 bp, Artificial Sequence
  Description of Artificial Sequence Synthetic oligonucleotide
ggaccttggcgtggccgtgc

SEQ ID NO: 68 - protein, 17 aa, Artificial Sequence
  Description of Artificial Sequence Synthetic peptide
CRLPEGDLGKEIEQKYD

SEQ ID NO: 69 - DNA, 29 bp, Artificial Sequence
  Description of Artificial Sequence Synthetic oligonucleotide
aaaggaatgacttccagctgacctgtctc

SEQ ID NO: 70 - DNA, 29 bp, Artificial Sequence
  Description of Artificial Sequence Synthetic oligonucleotide
aatcagctggaagtcattcctcctgtctc

SEQ ID NO: 71 - DNA, 29 bp, Artificial Sequence
  Description of Artificial Sequence Synthetic oligonucleotide
aagatcgtcgagatgtctactcctgtctc

SEQ ID NO: 72 - DNA, 29 bp, Artificial Sequence
  Description of Artificial Sequence Synthetic oligonucleotide
aaagtagacatctcgacgatccctgtctc

SEQ ID NO: 73 - DNA, 29 bp, Artificial Sequence
  Description of Artificial Sequence Synthetic oligonucleotide
aaggtccatctggttggtattcctgtctc

SEQ ID NO: 74 - DNA, 29 bp, Artificial Sequence
  Description of Artificial Sequence Synthetic oligonucleotide
aaaataccaaccagatggacccctgtctc

SEQ ID NO: 75 - DNA, 29 bp, Artificial Sequence
  Description of Artificial Sequence Synthetic oligonucleotide
aagctggactcctcctacacacctgtctc

SEQ ID NO: 76 - DNA, 29 bp, Artificial Sequence
  Description of Artificial Sequence Synthetic oligonucleotide
aatgtgtaggaggagtccagccctgtctc

SEQ ID NO: 77 - DNA, 29 bp, Artificial Sequence
  Description of Artificial Sequence Synthetic oligonucleotide
aaagtcgaccttcagtaaggacctgtctc

SEQ ID NO: 78 - DNA, 29 bp, Artificial Sequence
  Description of Artificial Sequence Synthetic oligonucleotide
aatccttactgaaggtcgactcctgtctc

SEQ ID NO: 79 - RNA, 20 nt, Artificial Sequence
  Description of Artificial Sequence Synthetic oligonucleotide
aagcuggacuccuccuacac

SEQ ID NO: 80 - RNA, 21 nt, Artificial Sequence
  Description of Artificial Sequence Synthetic oligonucleotide
aaacacauccuccucaggucg

SEQ ID NO: 81 - DNA, 21 bp, Artificial Sequence
  Description of Artificial Sequence Synthetic oligonucleotide
aaaggaatgacttccagctga

SEQ ID NO: 82 - DNA, 21 bp, Artificial Sequence
  Description of Artificial Sequence Synthetic oligonucleotide
aagatcgtcgagatgtctact

SEQ ID NO: 83 - DNA, 21 bp, Artificial Sequence
  Description of Artificial Sequence Synthetic oligonucleotide
aaggtccatctggttggtatt

SEQ ID NO: 84 - DNA, 21 bp, Artificial Sequence
  Description of Artificial Sequence Synthetic oligonucleotide
aagctggactcctcctacaca

SEQ ID NO: 85 - DNA, 21 bp, Artificial Sequence
  Description of Artificial Sequence Synthetic oligonucleotide
aaagtcgaccttcagtaagga

SEQ ID NO: 86 - DNA, 21 bp, Artificial Sequence
  Description of Combined DNA/RNA Molecule Synthetic oligonucleotide
  Description of Artificial Sequence Synthetic siRNA sequence
  modified_base (1)..(2): a, t, c, g or u
nngcuggacuccuccuacaca

SEQ ID NO: 87 - DNA, 21 bp, Artificial Sequence
  Description of Combined DNA/RNA Molecule Synthetic oligonucleotide
  Description of Artificial Sequence Synthetic siRNA sequence
gcuggacuccuccuacacatt

SEQ ID NO: 88 - DNA, 21 bp, Artificial Sequence
  Description of Artificial Sequence Synthetic siRNA sequence
uguguaggaggaguccagctt

SEQ ID NO: 89 - DNA, 21 bp, Artificial Sequence
  Description of Combined DNA/RNA Molecule Synthetic oligonucleotide
  Description of Artificial Sequence Synthetic siRNA sequence
  modified_base (1)..(2): a, t, c, g or u
nnacacauccuccucaggucg

SEQ ID NO: 90 - DNA, 21 bp, Artificial Sequence
  Description of Combined DNA/RNA Molecule Synthetic oligonucleotide
  Description of Artificial Sequence Synthetic siRNA sequence
acacauccuccucaggucgtt

SEQ ID NO: 91 - DNA, 21 bp, Artificial Sequence
  Description of Combined DNA/RNA Molecule Synthetic oligonucleotide
  Description of Artificial Sequence Synthetic siRNA sequence
cgaccugaggaggaugugutt

SEQ ID NO: 92 - DNA, 24 bp, Artificial Sequence
  Description of Artificial Sequence Synthetic siRNA sequence
cggatggcaacatttagaattagt

SEQ ID NO: 93 - DNA, 24 bp, Artificial Sequence
  Description of Artificial Sequence Synthetic Primer
tgattgagactgtaatcaagaacc

SEQ ID NO: 94 - DNA, 22 bp, Artificial Sequence
  Description of Artificial Sequence Synthetic Primer
ctgatgcccccatgttcgtcat

SEQ ID NO: 95 - DNA, 20 bp, Artificial Sequence
  Description of Artificial Sequence Synthetic Primer
ccaccaccctgttgctgtag

SEQ ID NO: 96 - DNA, 21 bp, Artificial Sequence
  Description of Combined DNA/RNA Molecule Synthetic oligonucleotide
  Description of Artificial Sequence Synthetic oligonucleotide
ttcgaccugaggaggaugugu

SEQ ID NO: 97 - DNA, 21 bp, Artificial Sequence
  Description of Combined DNA/RNA Molecule Synthetic oligonucleotide
  Description of Artificial Sequence Synthetic oligonucleotide
ttuccuuacugaaggucgacu

SEQ ID NO: 98 - DNA, 21 bp, Artificial Sequence
  Description of Combined DNA/RNA Molecule Synthetic oligonucleotide
  Description of Artificial Sequence Synthetic oligonucleotide
aggaaugacuuccagcugatt

SEQ ID NO: 99 - DNA, 21 bp, Artificial Sequence
  Description of Combined DNA/RNA Molecule Synthetic oligonucleotide
  Description of Artificial Sequence Synthetic oligonucleotide
ucagcuggaagucauuccutt

(19) United States
(12) Patent Application Publication
(10) Pub. No.: US 2007/0238691 A1
(43) Pub. Date: Oct. 11, 2007

(54) Inhibition of HIV replication and expression of p24 with eIF-5A

(75) Inventors: John E. Thompson, Waterloo (CA); Catherine Taylor, Waterloo (CA); Charles A. Dinarello, Denver, CO (US); Richard S. Dondero, Riverdale, NJ (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Senesco Technologies, Inc., New Brunswick, NJ (US)

(21) Appl. No.: 11/727,748

(22) Filed: Mar. 28, 2007

Related U.S. Application Data
(60) Provisional application No. 60/786,806, filed on Mar. 29, 2006.

(51) Int. Cl. A61K 48/00

(52) U.S. Cl. 514/44

(57) ABSTRACT

The present invention relates to methods of inhibiting the replication of the HIV virus by providing siRNA or antisense polynucleotides of eIF-5A1. The present invention also provides methods of inhibiting expression of p24 with siRNA or antisense polynucleotides of eIF-5A1.

This application claims priority to U.S. provisional application 60/786,806, filed on Mar. 29, 2006, which is herein incorporated by reference in its entirety.

### FIELD OF THE INVENTION

The present invention relates to methods of inhibiting HIV replication and expression of p24 using siRNA or antisense constructs of apoptosis-specific eucaryotic initiation factor (“eIF-5A”) or referred to as “apoptosis-specific eIF-5A” or “eIF-5A1.”

### BACKGROUND OF THE INVENTION

HIV primarily infects cells with CD4 cell-surface receptor molecules, using them to gain entry. Many cell types share common epitopes with this protein, though CD4 lymphocytes play a crucial role. In macrophages and in some other cells lacking CD4 receptors, such as fibroblasts, an Fc receptor site or complement receptor site may be used instead for entry of HIV. Cells of the mononuclear phagocyte system, principally blood monocytes and tissue macrophages, T lymphocytes, B lymphocytes, natural killer (NK) lymphocytes, dendritic cells (Langerhans cells of epithelia and follicular dendritic cells in lymph nodes), hematopoietic stem cells, endothelial cells, microglial cells in brain, and gastrointestinal epithelial cells are the primary targets of HIV infection.

The mature virus consists of a bar-shaped electron dense core containing the viral genome—two short strands of ribonucleic acid (RNA) about 9200 nucleotide bases long—along with the enzymes reverse transcriptase, protease, ribonuclease, and integrase, all encased in an outer lipid envelope with 72 surface projections containing an antigen, gp120, that aids in the binding of the virus to the target cells with CD4 receptors. By electron microscopy, the plasma membrane of an infected CD4+ lymphocyte exhibits budding virus particles approximately 90 to 100 n in diameter. The genome of HIV, similar to retroviruses in general, contains three major genes—gag, pol, and env.

The major structural components coded by env include the envelope glycoproteins, including the outer envelope glycoprotein gp120 and transmembrane glycoprotein gp41 derived from glycoprotein precursor gp160. Major components coded by the gag gene include core nucleocapsid proteins p55, p40, p24 (capsid, or “core” antigen), p17 (matrix), and p7 (nucleocapsid); the important proteins coded by pol are the enzyme proteins p66 and p51 (reverse transcriptase), p11 (protease), and p32 (integrase). Although most of the major HIV viral proteins, which include p24 (core antigen) and gp41 (envelope antigen), are highly immunogenic, the antibody responses vary according to the virus load and the immune competence of the host. The antigenicity of these various components provides a means for detection of antibody, the basis for most HIV testing.

HIV has the additional ability to mutate easily, in large part due to the error rate of the reverse transcriptase enzyme, which introduces a mutation approximately once per 2000 incorporated nucleotides. This high mutation rate leads to the emergence of HIV variants within the infected person's cells that can resist immune attack, are more cytotoxic, can generate syncytia more readily, or can resist drug therapy. Over time, different tissues of the body may harbor differing HIV variants.

After entering the body, the viral particle is attracted to a cell with the appropriate CD4 receptor molecules where it attaches by fusion to a susceptible cell membrane or by endocytosis and then enters the cell. The probability of infection is a function of both the number of infective HIV virions in the body fluid which contacts the host as well as the number of cells available at the site of contact that have appropriate CD4 receptors.

Within the cell, the viral particle uncoats from the envelope to releases its RNA. The enzyme product of the pol gene, reverse transcriptase that is bound to the HIV RNA, provides for reverse transcription of RNA to proviral DNA. It is this HIV proviral DNA which is then inserted into host cell genomic DNA by the integrase enzyme. Once the HIV proviral DNA is within the infected cell's genome, it cannot be eliminated or destroyed except by destroying the cell itself. The HIV provirus is then replicated by the host cell. The infected cell can then release virions by surface budding, or infected cells can undergo lysis with release of new HIV virions which can then infect additional cells. Antibodies formed against HIV are not protective, and a viremic state can persist despite the presence of even high antibody titers.

After initial entry of HIV and establishment of infection, replication may at first occur within inflammatory cells at the site of infection or within peripheral blood mononuclear cells, but then the major site of replication quickly shifts to lymphoid tissues of the body, including those in lymph nodes, spleen, liver, and bone marrow. Besides lymph nodes, the gut associated lymphoid tissue provides a substantial reservoir for HIV.

Macrophages and Langerhans cells in epithelia such as in the genital tract are important both as reservoirs and vectors for the spread of HIV in the body. Langerhans cells (a subset of blood dendritic cells) act as antigen presenting cells for CD4 lymphocytes. Both macrophages and Langerhans cells can be HIV-infected but are not destroyed themselves. HIV can then be carried elsewhere in the body. Within lymph nodes, HIV virions are trapped in the processes of follicular dendritic cells (FDC's), where they may infect CD4 lymphocytes that are percolating through the node. The FDC's themselves become infected, but are not destroyed.

Viral replication is stimulated by a variety of cytokines such as interleukins and tumor necrosis factor which activate CD4 lymphocytes and make them more susceptible to HIV infection. Primary HIV infection is followed by a burst of viremia in which virus is easily detected in peripheral blood in mononuclear cells and plasma. In the period of clinical latency of HIV infection, there is little detectable virus in peripheral blood, but viral replication actively continues in lymphoid tissues.

Subsets of the CD4+ lymphocyte population are important in determining the host response to infection. The subset known as TH1 (T helper 1) is responsible for directing a cytotoxic CD8 lymphocyte (CTL) response, but the TH2 (T helper 2) subset of CD4 and CD8 T-lymphocytes diminishes the CTL response while increasing antibody production. HIV-infections accompanied by a dominant TH1 response tend to proceed longer. The switch from a TH1 to a TH2 response has been suggested as a factor in the development of AIDS, but not all cytokines in HIV-infected persons at different stages of disease corroborate this hypothesis. Production of interleukin-5 and interferon-gamma by CD4 and CD8 lymphocytes expressing CD30, however, is associated with promotion of B-lymphocyte immunoglobulin production.

The primary target of HIV is the immune system itself, which is gradually destroyed. Viral replication actively continues following initial HIV infection, and the rate of CD4 lymphocyte destruction is progressive. Clinically, HIV infection may appear “latent” for years during this period of ongoing immune system destruction. During this time, enough of the immune system remains intact to provide immune surveillance and prevent most infections. Eventually, when a significant number of CD4 lymphocytes have been destroyed and when production of new CD4 cells cannot match destruction, then failure of the immune system leads to the appearance of clinical AIDS.

Infection with HIV is sustained through continuous viral replication with reinfection of additional host cells. Both HIV in host plasma and HIV-infected host cells appear to have a short lifespan; and late in the course of AIDS the half-life of plasma HIV is only about 2 days. Thus, the persistent viremia requires continuous reinfection of new CD4 lymphocytes followed by viral replication and infected host cell turnover. This rapid turnover of HIV and CD4 lymphocytes promotes origin of new strains of HIV within the host from mutation of HIV.

Presence or emergence of different HIV subtypes may also account for the appearance of antiretroviral drug resistance as well as the variability in pathologic lesions as different cell types are targeted or different cytopathic effects are elicited during the course of infection. Phylogenetic studies can identify genetic clusters of HIV-1 env genes which are known as subtypes, or clades, that have arisen with progression of the AIDS epidemic worldwide. The V3 loop amino acid sequences of these genetic variants influence HIV phenotype and immune response. Thus, the biologic properties of HIV can vary, even within an individual HIV infected person, where variants of HIV may arise that are “neurotropic” or “lymphocytotropic” for example.

Despite evidence that prevention programs instituted some time ago are beginning to have an impact in some countries, the HIV/AIDS epidemic continues to grow. By the end of 2005, 40.3 million people were living with HIV/AIDS, including 17.5 million women and 2.3 million children under the age of 15. 4.9 million people became newly infected with HIV in 2005, including 700,000 children. Of these, 3.2 million new infections occurred in Sub-Saharan Africa. In 2005 alone, a total of 3.1 million people died of HIV/AIDS-related causes. World-wide, only one in ten persons infected with HIV has been tested and knows his/her HIV status.

There are now many U.S. government-approved HIV medications. However, none of these medications can cure HIV, and no single drug taken alone is effective. But, taken in a combination of at least three, these medications can control the quantity of virus in the body and maintain the health of the immune system. This combination is called Highly Active Anti-Retroviral Therapy, or HAART. HIV medications fall into four types or “classes”: NRTIs (nucleoside or nucleotide reverse transcriptase inhibitors) (e.g. Emtriva™, Epivir®, Epzicom™, Truvada®, Videx®, Viread®, Ziagen®, Combivir®, Retrovir®, Trizivir®, Zerit®); NNRTIs (non-nucleoside reverse transcriptase inhibitors) (e.g. Sustiva®, Viramune®,); PIs (protease inhibitors) (e.g. Kaletra®, Lexiva®, Reyataz®, Aptivus®, Crixivan®, Invirase®, Norvir®, Viracept®); and Fusion inhibitors (e.g. Fuzeon®).

All four classes of medications have been designed to interfere with HIV's ability to copy itself—that is, to reproduce inside the body. Each class of medication stops the virus at a different moment in its reproductive cycle.

Doctors have not yet discovered a single combination of HIV medications that's best for everyone. Each combination has its advantages and disadvantages. Unfortunately, researchers can't compare the hundreds of possible combinations of individual medications. Instead, they usually try to compare combinations of classes of medications. Three class combinations are commonly researched and prescribed today for people starting HIV treatment: One NNRTI plus two NRTIs; one or two PIs plus two NRTIs; or one “boosted” PI plus two NRTIs.

Thus, although treatment regimens are available, none of these to date “cure” or completely wipe out the presence of HIV in the body. In addition, over time, the HIV may develop a resistance to these medicines. Accordingly, there remains a need for an alternative and additional new therapy that can be used alone or conjunction with the previously described therapies/drugs. The present invention fulfills this need by providing a method of inhibiting HIV replication with siRNA or antisense constructs of apoptosis-specific eucaryotic translation initiation Factor-5A (eIF-5A or eIF-5A1).

eIF-5A and deoxyhypusine synthase (DHS) are known to play important roles in many cellular processes including cell growth and differentiation. Hypusine, a unique amino acid, is found in all examined eucaryotes and archaebacteria, but not in eubacteria, and eIF-5A is the only known hypusine-containing protein. Park (1988) J. Biol. Chem., 263, 7447-7449; Schümann & Klink (1989) System. Appl. Microbiol., 11, 103-107; Bartig et al. (1990) System. Appl. Microbiol., 13, 112-116; Gordon et al. (1987a) J. Biol. Chem., 262, 16585-16589. Active eIF-5A is formed in two post-translational steps: the first step is the formation of a deoxyhypusine residue by the transfer of the 4-aminobutyl moiety of spermidine to the α-amino group of a specific lysine of the precursor eIF-5A catalyzed by deoxyhypusine synthase; the second step involves the hydroxylation of this 4-aminobutyl moiety by deoxyhypusine hydroxylase to form hypusine.

The amino acid sequence of eIF-5A is well conserved between species, and there is strict conservation of the amino acid sequence surrounding the hypusine residue in eIF-5A, which suggests that this modification may be important for survival. Park et al. (1993) Biofactors, 4, 95-104. This assumption is further supported by the observation that inactivation of both isoforms of eIF-5A found to date in yeast, or inactivation of the DHS gene, which catalyzes the first step in their activation, blocks cell division. Schnier et al. (1991) Mol. Cell. Biol., 11, 3105-3114; Sasaki et al. (1996) FEBS Lett., 384, 151-154; Park et al. (1998) J. Biol. Chem., 273, 1677-1683. However, depletion of eIF-5A protein in yeast resulted in only a small decrease in total protein synthesis suggesting that eIF-5A may be required for the translation of specific subsets of mRNA's rather than for protein global synthesis. Kang et al. (1993), “Effect of initiation factor eIF-5A depletion on cell proliferation and protein synthesis,” in Tuite, M. (ed.), Protein Synthesis and Targeting in Yeast, NATO Series H. The recent finding that ligands that bind eIF-5A share highly conserved motifs also supports the importance of eIF-5A. Xu & Chen (2001) J. Biol. Chem., 276, 2555-2561. In addition, the hypusine residue of modified eIF-5A was found to be essential for sequence-specific binding to RNA, and binding did not provide protection from ribonucleases.

In addition, intracellular depletion of eIF-5A results in a significant accumulation of specific mRNAs in the nucleus, indicating that eIF-5A may be responsible for shuttling specific classes of mRNAs from the nucleus to the cytoplasm. Liu & Tartakoff (1997) Supplement to Molecular Biology of the Cell, 8, 426a. Abstract No. 2476, 37th American Society for Cell Biology Annual Meeting. The accumulation of eIF-5A at nuclear pore-associated intranuclear filaments and its interaction with a general nuclear export receptor further suggest that eIF-5A is a nucleocytoplasmic shuttle protein, rather than a component of polysomes. Rosorius et al. (1999) J. Cell Science, 112, 2369-2380.

The first cDNA for eIF-5A was cloned from human in 1989 by Smit-McBride et al., and since then cDNAs or genes for eIF-5A have been cloned from various eukaryotes including yeast, rat, chick embryo, alfalfa, and tomato. Smit-McBride et al. (1989) J. Biol. Chem., 264, 1578-1583; Schnier et al. (1991) (yeast); Sano, A. (1995) in Imahori, M. et al. (eds), Polyamines, Basic and Clinical Aspects, VNU Science Press, The Netherlands, 81-88 (rat); Rinaudo & Park (1992) FASEB J., 6, A453 (chick embryo); Pay et al. (1991) Plant Mol. Biol., 17, 927-929 (alfalfa); Wang et al. (2001) J. Biol. Chem., 276, 17541-17549 (tomato).

Expression of eIF-5A mRNA has been explored in various human tissues and mammalian cell lines. For example, changes in eIF-5A expression have been observed in human fibroblast cells after addition of serum following serum deprivation. Pang & Chen (1994) J. Cell Physiol., 160, 531-538. Age-related decreases in deoxyhypusine synthase activity and abundance of precursor eIF-5A have also been observed in senescing fibroblast cells, although the possibility that this reflects averaging of differential changes in isoforms was not determined. Chen & Chen (1997) J. Cell Physiol., 170, 248-254.

Studies have shown that eIF-5A1 may be the cellular target of viral proteins such as the human immunodeficiency virus type 1 Rev protein and human T cell leukemia virus type 1 Rex protein. Ruhl et al. (1993) J. Cell Biol., 123, 1309-1320; Katahira et al. (1995) J. Virol., 69, 3125-3133. Preliminary studies indicate that eIF-5A1 may target RNA by interacting with other RNA-binding proteins such as Rev, suggesting that these viral proteins may recruit eIF-5A1 for viral RNA processing. Liu et al. (1997) Biol. Signals, 6, 166-174.

### SUMMARY OF INVENTION

The present invention relates to methods of inhibiting the replication of the HIV virus by providing siRNA or antisense polynucleotides of apoptosis specific eucaryotic initiation factor 5A (eIF-5A), referred to as “apoptosis-specific eIF-5A” or “eIF-5A1.” The present invention also provides methods of inhibiting expression of p24 with siRNA or antisense polynucleotides of eIF-5A1.

The present invention provides a method for inhibiting replication of HIV virus comprising inhibiting expression of eIF-5A1 with an agent capable of inhibiting expression of eIF-5A1 wherein the inhibition of expression of eIF-5A1 inhibits expression of p24 and thereby inhibits HIV replication. The agent may be an antisense polynucleotide of eIF-5A1 or an siRNA targeted at eIF-5A1. Exemplary antisense polynucleotides are SEQ ID NO: 63, SEQ ID NO: 64, SEQ ID NO: 65; SEQ ID NO:25, SEQ ID NO:26, and SEQ ID NO:27. Exemplary siRNAs are SEQ ID NO: 46, SEQ ID NO: 48, SEQ ID NO: 51, and SEQ ID NO: 54, or siRNAs having the sequence of dTdTCGACCUGAGGAGGAUGUGU or dTdTUCCUUACUGAAGGUCGACU.

The present invention also provides a method of inhibiting expression of p24 in a HIV infected cell, comprising inhibiting expression of eIF-5A1 with any of the agents discussed herein.

The present invention also provides pharmaceutical compositions useful for inhibiting HIV replication or inhibiting p24 expression in an HIV infected cell. The compositions comprise any of the antisense polynucleotides or siRNAs discussed herein.

### BRIEF DESCRIPTION OF THE DRAWINGS

FIG. 1 depicts the nucleotide sequence of rat corpus luteum eIF-5A1 full-length cDNA (SEQ ID NO: 1). The amino acid sequence is shown in SEQ ID NO: 2.

FIG. 2 is an alignment of the full-length nucleotide sequence of rat corpus luteum eIF-5A1 cDNA (SEQ ID NO: 20) with the nucleotide sequence of human eIF-5A1 (SEQ ID NO: 3) (Accession number BC000751 or NM_{—}001970, SEQ ID NO:3).

FIG. 3 is an alignment of the full-length nucleotide sequence of rat corpus luteum eIF-5A1 cDNA (SEQ ID NO: 20) with the nucleotide sequence of human eIF-5A1 (SEQ ID NO: 4) (Accession number NM-020390, SEQ ID NO:4).

FIG. 4 is an alignment of the full-length nucleotide sequence of rat corpus luteum eIF-5A1 cDNA (SEQ ID NO: 20) with the nucleotide sequence of mouse eIF-5A1 (Accession number BC003889). Mouse nucleotide sequence (Accession number BC003889) is SEQ ID NO:5.

FIG. 5 is an alignment of the derived full-length amino acid sequence of rat corpus luteum eIF-5A1 (SEQ ID NO: 2) with the derived amino acid sequence of human eIF-5A (SEQ ID NO: 21) (Accession number BC000751 or NM_{—}001970).

FIG. 6 is an alignment of the derived full-length amino acid sequence of rat corpus luteum eIF-5A1 (SEQ ID NO: 2) with the derived amino acid sequence of human eIF-5A (SEQ ID NO: 22) (Accession number NM_{—}020390).

FIG. 7 is an alignment of the derived full-length amino acid sequence of rat corpus luteum eIF-5A1 (SEQ ID NO: 2) with the derived amino acid sequence of mouse eIF-5A1 (SEQ ID NO: 23) (Accession number BC003889).

FIG. 8 shows the sequence of human eIF-5A1 (SEQ ID NO:29) and the target sequences of 5 siRNAs (SEQ ID NO:30, 31, 32, and 33).

FIG. 9 shows the sequence of human eIF-5A1 (SEQ ID NO: 29) and the sequences of 3 antisense oligonucleotides (SEQ ID NO:63, 64, and 65), respectively in order of appearance).

FIG. 10 shows the binding position of three antisense oligonucleotides (SEQ ID NO:25-27, respectively in order of appearance) targeted against human eIF-5A1. The full-length nucleotide sequence is SEQ ID NO: 19.

FIGS. 11a and b show the nucleotide alignment (SEQ ID NO: 41 and 42, respectively in order of appearance) and amino acid alignment (SEQ ID NO: 43 and 22, respectively in order of appearance) of human eIF-5A1 against human proliferating eIF-5A.

FIG. 12 depicts the design of siRNAs against eIF-5A1. The siRNAs have the SEQ ID NO: 46, 49, 52, and 55. The full-length nucleotide sequence is shown in SEQ ID NO: 29.

FIG. 13 shows that eIF-5A1 is not required for cell proliferation.

FIG. 14 is a model of eIF5-A1 function and regulation. In healthy cells, eIF-5A1 is hypusinated by DHS and localized in the cytoplasm. Hypusinated eIF-5A1 may support cell growth via some unknown cytoplasmic function. Genotoxic stress or death receptor activation stimulate translocation of eIF-5A1 into the nucleus where it participates in the induction or execution of apoptotic cell death. In the event of apoptosis induced by genotoxic stress, nuclear eIF-5A1 may function to regulate the expression of p53, possibly by regulating the nuclear export of its mRNA.

FIG. 15 shows the results of an XTT cell proliferation assay. The results show that siRNA against eIF-5A1 (eIF-5A1) does not inhibit cell division. siRNA directed against cell proliferation eIF-5A (eIF-5A2) inhibits cell division.

FIG. 16 shows that siRNA of eIF-5A1 inhibits p24 expression in stimulated and unstimulated U1 cells.

FIG. 17 shows that shows that siRNA of eIF-5A1 inhibits I1-8 expression in stimulated and unstimulated U1 cells.

### DETAILED DESCRIPTION OF THE INVENTION

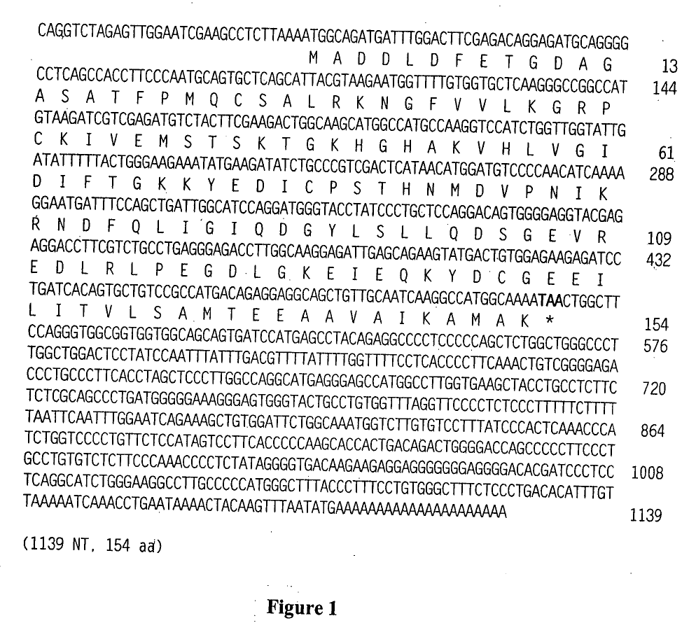

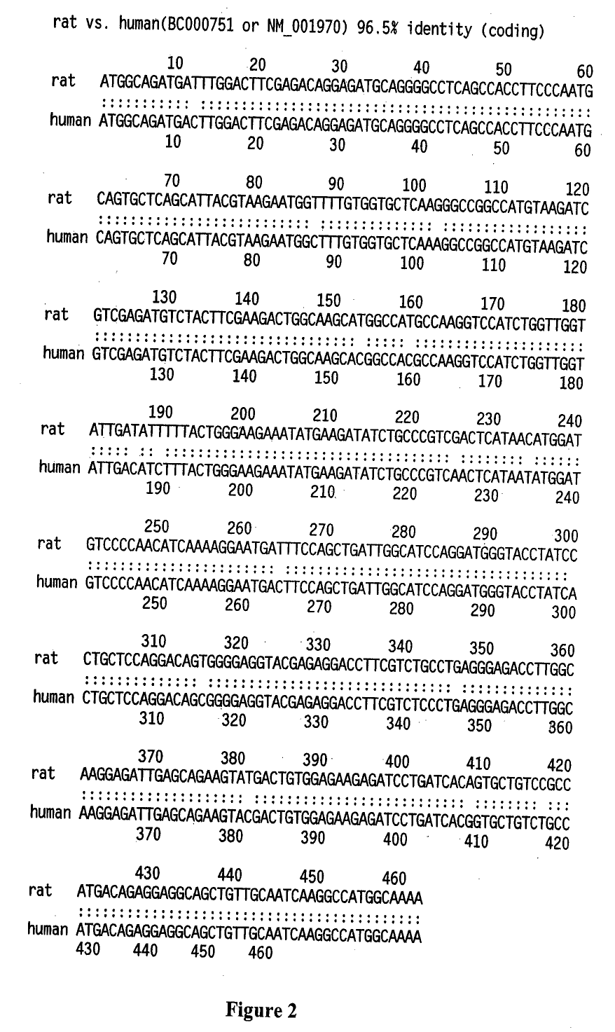

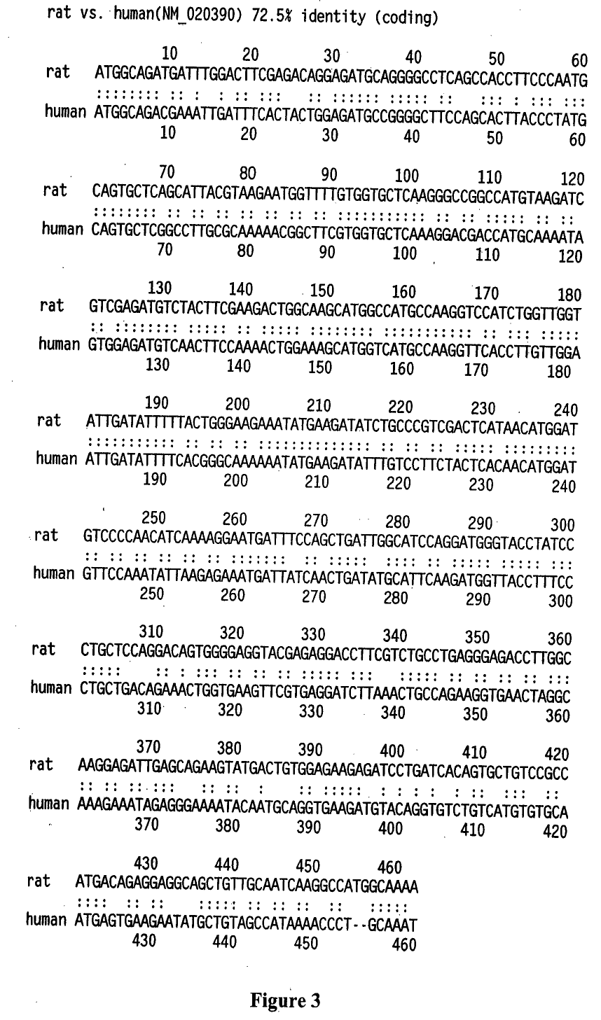

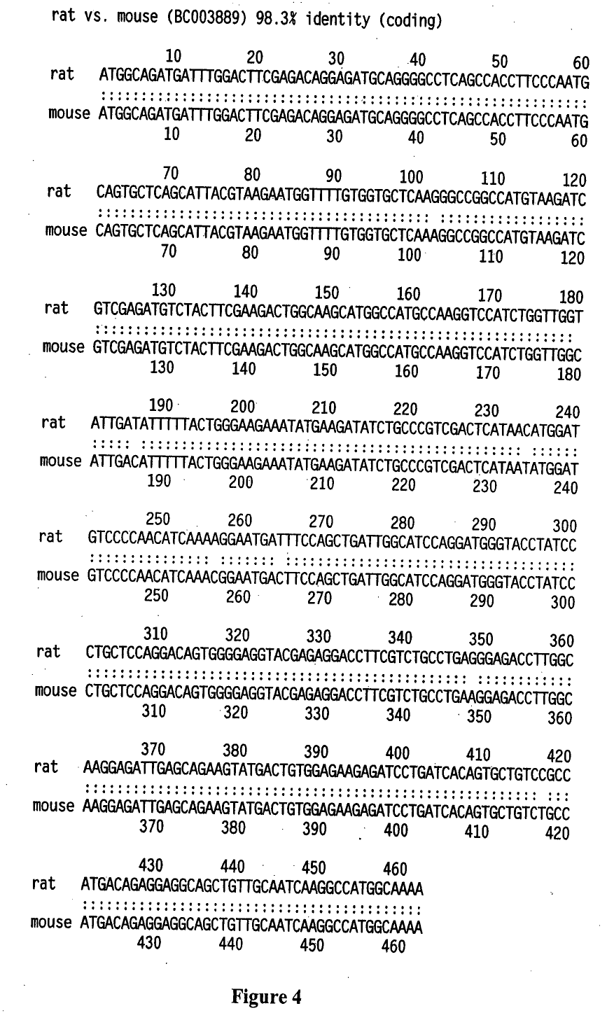

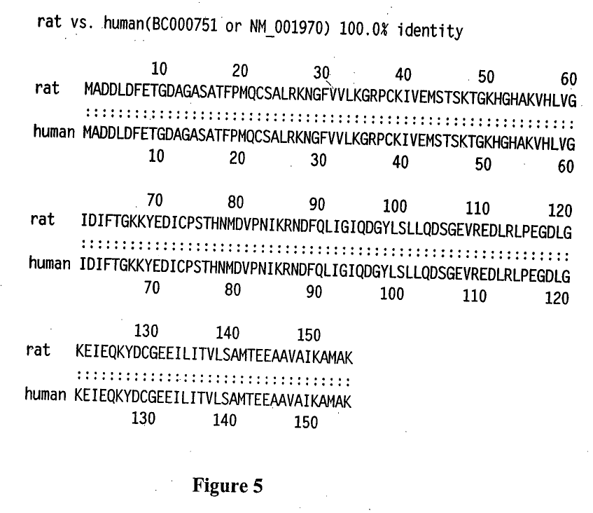

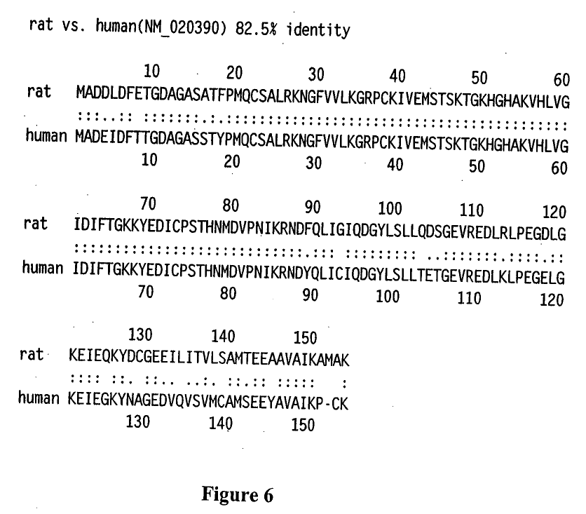

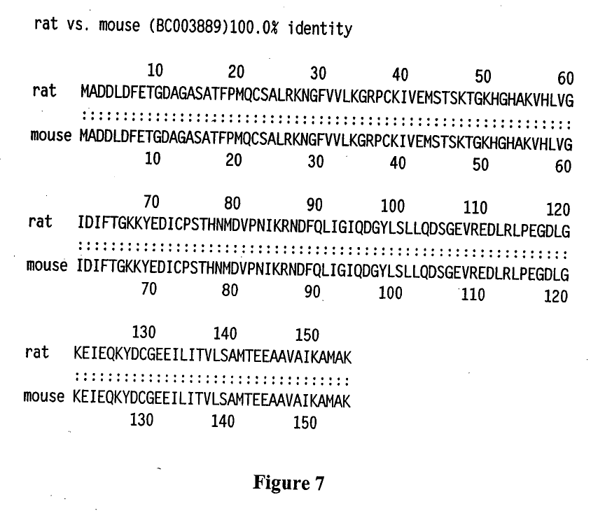

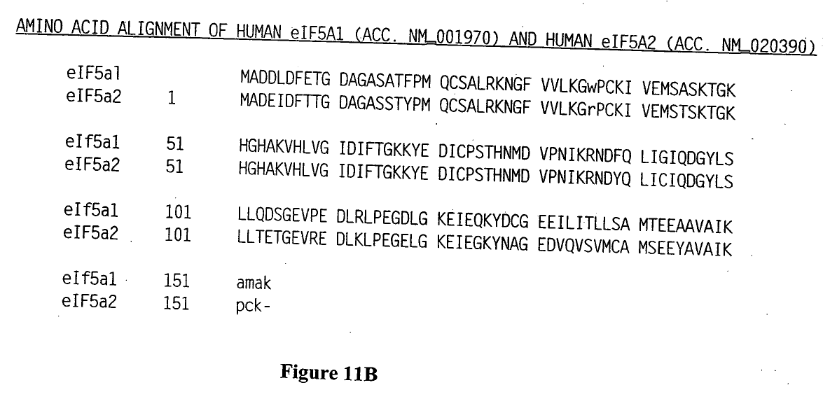

Several isoforms of eukaryotic initiation factor 5A (“eIF-5A”) have been isolated and present in published databanks. It was thought that these isoforms were functionally redundant. The present inventors have discovered that one isoform is upregulated immediately before the induction of apoptosis, which they have designated apoptosis-specific eIF-5A or eIF-5A1. The subject of the present invention utilizes eIF-5A1. FIGS. 1-7 show the sequence (nucleotide and amino acid) of rat, mouse and human eIF-5A1. The other isoform is believed to be involved in cellular proliferation and is named “proliferation eIF-5A” or “eIF-5A2.” FIG. 11 shows the comparison of eIF-5A1 with eIF-5A2.

The present invention provides a method of inhibiting HIV replication in a cell comprising transfecting a cell infected with HIV with an siRNA or antisense polynucleotide of eIF-5A1. The siRNA or antisense polynucleotide inhibits expression eIF-5A, which in turn inhibits expression of p24. By inhibiting expression of p24, HIV replication is inhibited.

Inhibition of eIF-5A1 expression with antisense polynucleotides and siRNAs to eIF-5A1 has been demonstrated by the present inventors in co-pending applications (Ser. No. 11/293,391, filed Dec. 5, 2005; Ser. No. 11/184,982 filed on Jul. 20, 2005; Ser. No. 11/595,990 filed Nov. 13, 2006; Ser. No. 11/637,835 filed Dec. 13, 2006; Ser. No. 11/______ filed Mar. 20, 2007 entitled “Use of Apoptosis-specific eIF-5A siRNA to Down Regulate Expression of Proinflammatory Cytokines to Treat Sepsis” and Ser. No. 11/______ filed Mar. 20, 2007 entitled “A Novel Method Of Protecting Islet Cells From Apoptosis During The Donor Harvesting Process”), which are herein incorporated by reference in its entirety.

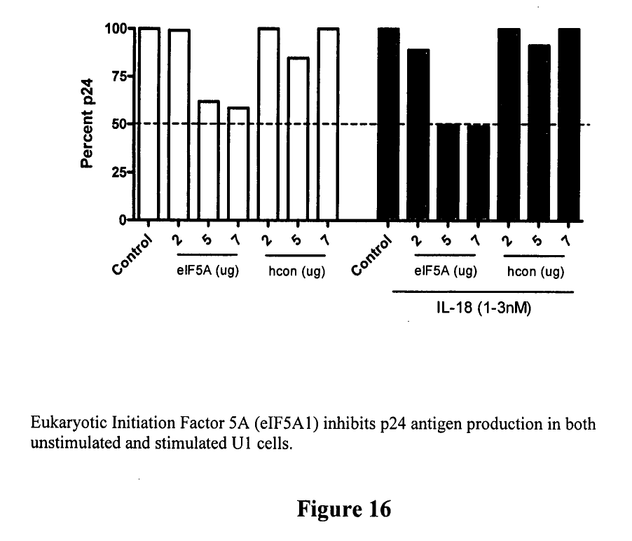

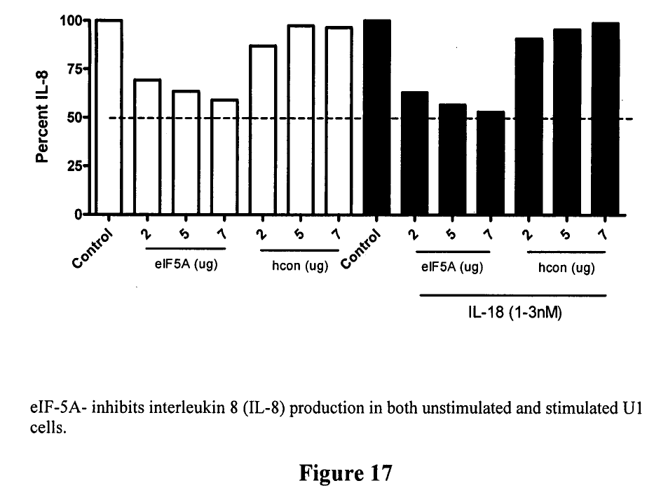

The present inventors have shown that siRNAs to eIF-5A1 reduce the amount of p24 and IL-8 each by approximately 50 percent in HIV-infected cells. See Examples 1 and 2 and FIGS. 16 and 17. The levels of p24, a core protein in HIV cells, and IL-8, a proinflammatory cytokine, rise proportionately with increased HIV replication making both of them standard indicators of HIV-1 replication.

A chronically HIV-1 infected human cell line was transfected with a small interfering RNA (“siRNA”) to eIF-5A1 and then levels of p24 or IL-8 were measured, in separate assays, 72 hours later. The results show that eIF-5A1 siRNAs reduced levels of p24 and IL-8. It is known and understood in the art that reduced levels of p24 and 11-8 correlate proportionately to suppressed replication of HIV-1.

Since eIF-5A1 is a molecule made by the cell and not the virus, the eIF-5A1 does not mutate (as other proteins of the virus) and thus, an anti-HIV strategy using this target may not be hampered by resistance brought about by viral mutation, which is seen with traditional HIV therapies.

While not being bound by theory, it is believed that hypusinated eIF-5A1 assists the HIV rev protein in shuttling mRNAs needed for viral replication out of the nucleus. siRNA or antisense polynucleotides of eIF-5A1 knock down the levels of eIF-5A1, which in turn hinders the shuttling of select viral mRNAs (such as p24).

The Rev protein of HIV-1 is essential for viral replication. Rev binds to viral transcripts at the Rev Response Element (RRE) and shuttles the transcripts from the nucleus to the cytoplasm. Rev is required for the nuclear export and subsequent translation of viral transcripts encoding such key viral proteins as Gag (measured as p24), Pol, Env, Vif, Vpr, and Vpu (Reviewed by Culim and Malim Trends Biochem Sci; 16: pp. 346-350). In the absence of Rev, HIV-1 transcripts are retained in the cytoplasm preventing the expression of viral structural proteins and the generation of infective progeny.

Eukaryotic translation initiation factor 5A1 has been identified as a cellular co-factor of Rev and the presence of eIF5-A1 is required for proper Rev function (Ruhl et al. 1993). Expression levels of eIF5-A1 have also been found to be significantly upregulated in the PBMCs of HIV-1-infected donors, an observation consistant with a role for eIF-5A1 in HIV-1 replication (Bevec et al., Proc Natl Acad Sci USA.; 91:10829-33 (1994)). Similarly, eIF5-A1 has been found to be an essential co-factor for the viral transactivator protein Rex of human T-cell leukemia virus type I (Katahira et al., J Virol.; 69(5):3125-33 (1995)). Antisense suppression of eIF-5A1 (Ruhl et al., J. Cell Biol.; 123:1309-1320 (1993)) or over-expression of non-functional eIF5A mutants (Bevec et al., Science; 271:1858-60 (1996); Junker et al., Human Gene Ther.; 7:1861-9 (1996)) has been shown to inhibit HIV-1 replication, confirming an essential role for eIF-5A1 as a host co-factor in HIV-1 replication. Inhibitors of DHS (Hauber et al., J. Clin Invest.; 115(1): 76-85 (2005)) and DHH (Andrus et al., Biochem Pharmacol.; 55(11):1807-18 (1998)) have also been observed to have antiviral activity in HIV-1 studies. eIF5A has been proposed to act by targeting Rev/Rex-viral transcript complexes to the nuclear pore complex for subsequent nuclear export via the CRM1/exportin 1 nuclear export machinery (Elfgang et al., Proc Natl Acad Sci USA.; 96(11):6229-34 (1996); Hofmann et al., J Cell Biol.; 152(5):895-910 (2001)). In the absence of eIF5A, Rev/Rex cannot access the nuclear export machinery and consequently its viral transcript cargo is retained in the nucleus and unable to be translated into the viral proteins required for generating infective progeny.

As used herein, “inhibiting” means an inhibition, reduction or suppression of expression and refers to the absence or detectable decrease in the level of protein and/or mRNA product from a target gene, such as eIF-5A1, p24 or I1-8.

The siRNA or antisense polynucleotides may be any siRNA or antisense polynucleotides that inhibit expression of eIF-5A1. The eIF-5A1 may be from any species such as any mammal (including human), birds, etc. Exemplary eIF-5A1 include eIF-5A1 encoded by the nucleic acid sequences shown in SEQ ID NO: 1, 20, 3, 4, 5 or 29 or comprising the amino acid sequence set forth in SEQ ID NO: 2, 21, or 23. Also included is any eIF-5A1 nucleotide sequence has at least 70 percent homology with SEQ ID NO: 1, 20, 3, 4, 5 or 29 or an amino acid sequencing having at least 70 percent homology with 2, 21, or 23. As discussed herein, eIF-5A1 share a high degree of homology between species and are especially highly conserved in the hypusination region.

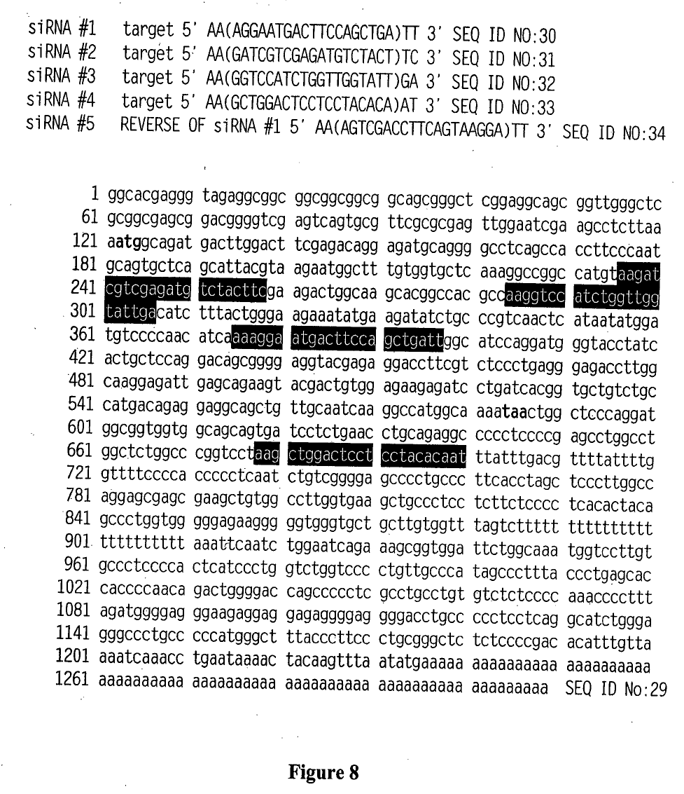

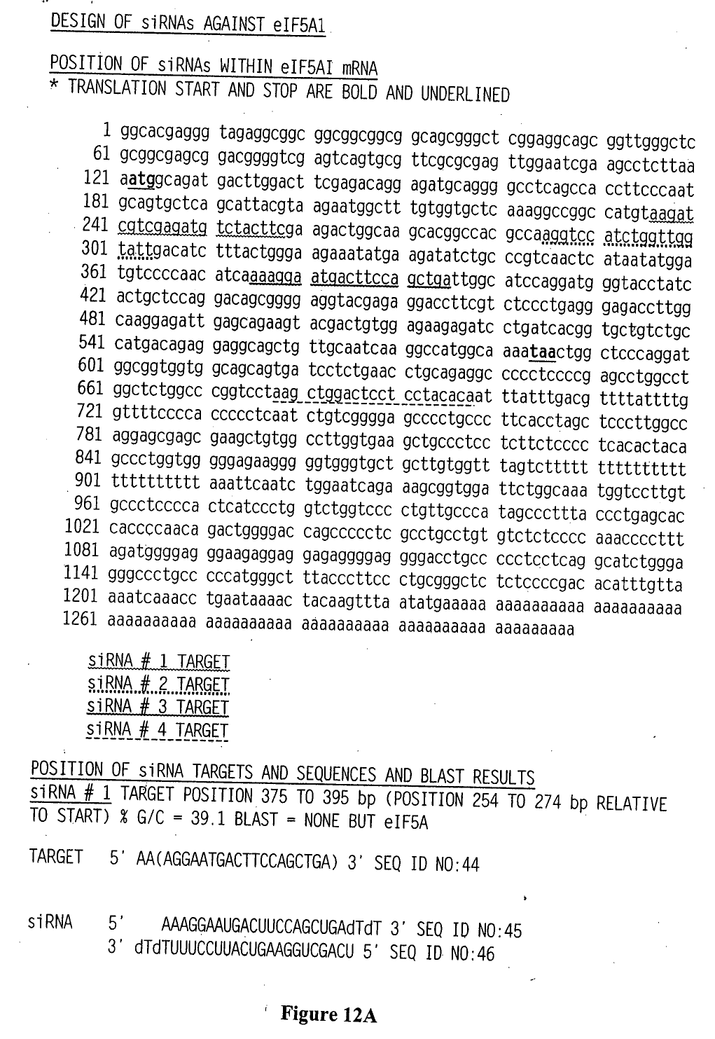

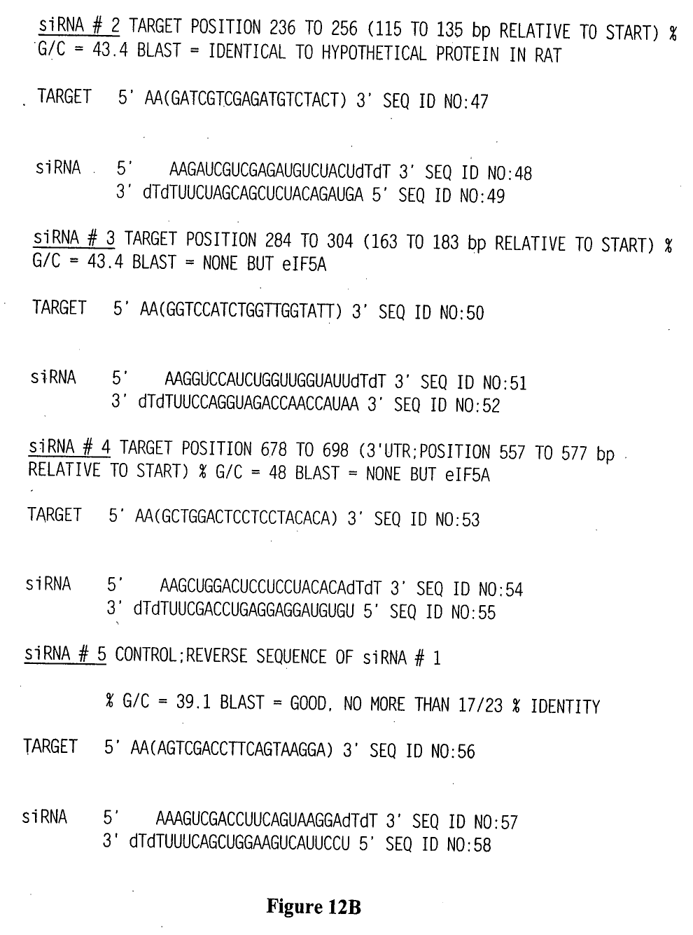

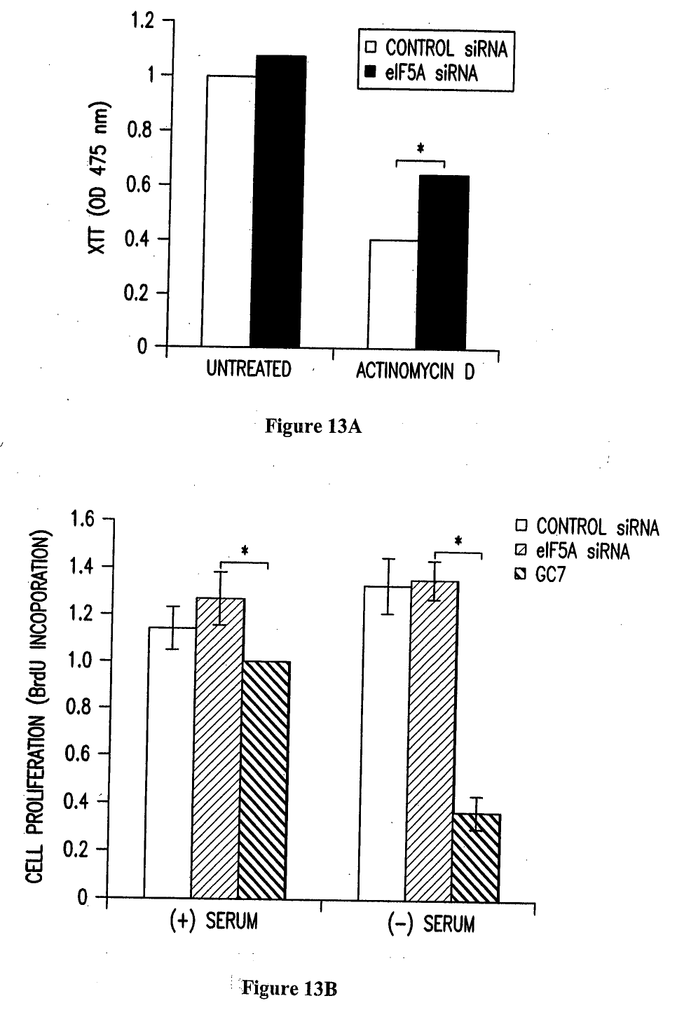

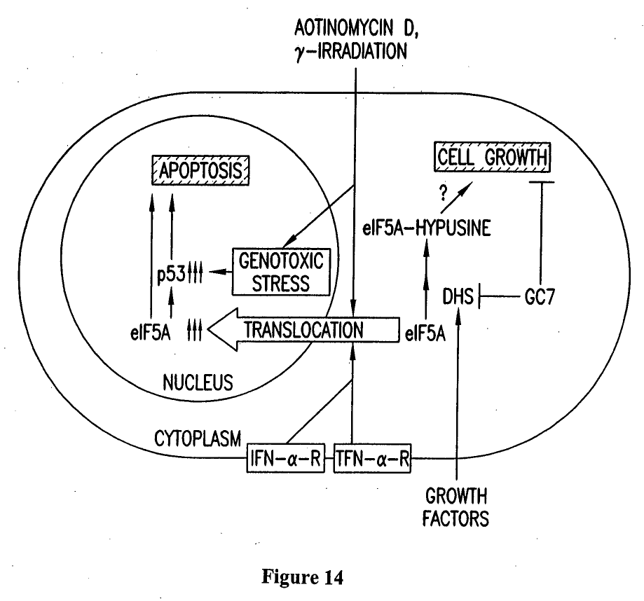

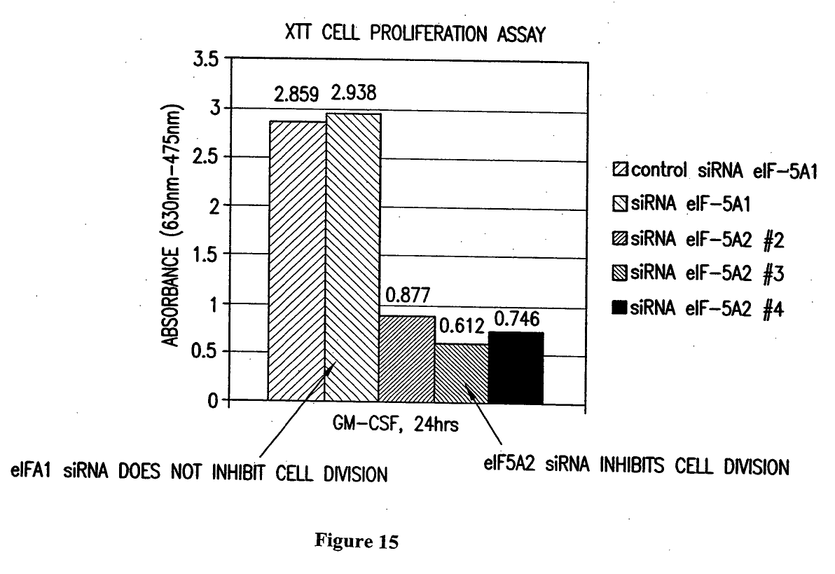

Exemplary siRNAs include an siRNA comprising the following nucleic acid sequence: 5′-GCUGGACUCCUCCUACACAdTdT-3′ and an siRNA comprises the following nucleic acid sequence: 5′-AGGAAUGACUUCCAGCUGAdTdT-3′. Other siRNAs are shown in FIGS. 8 and 12.

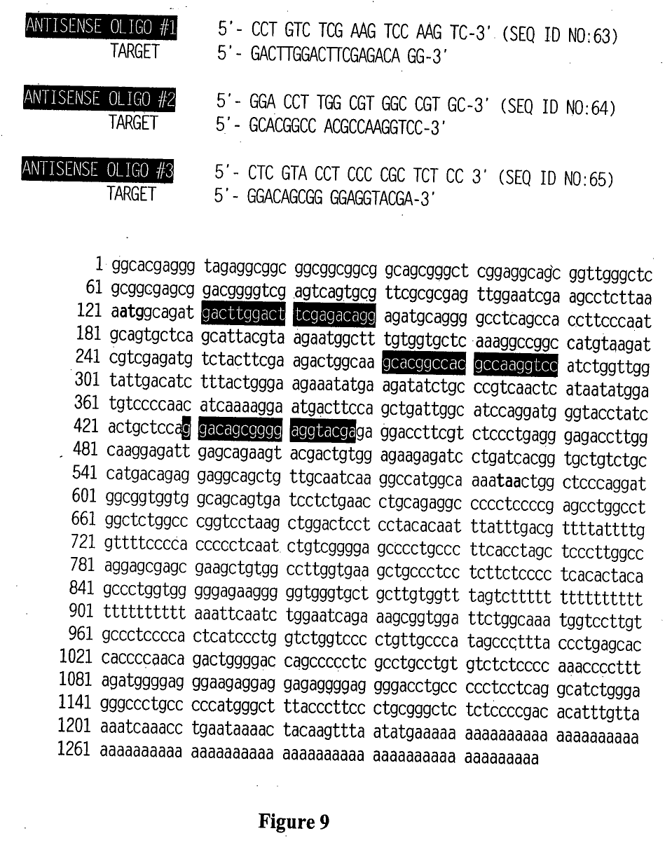

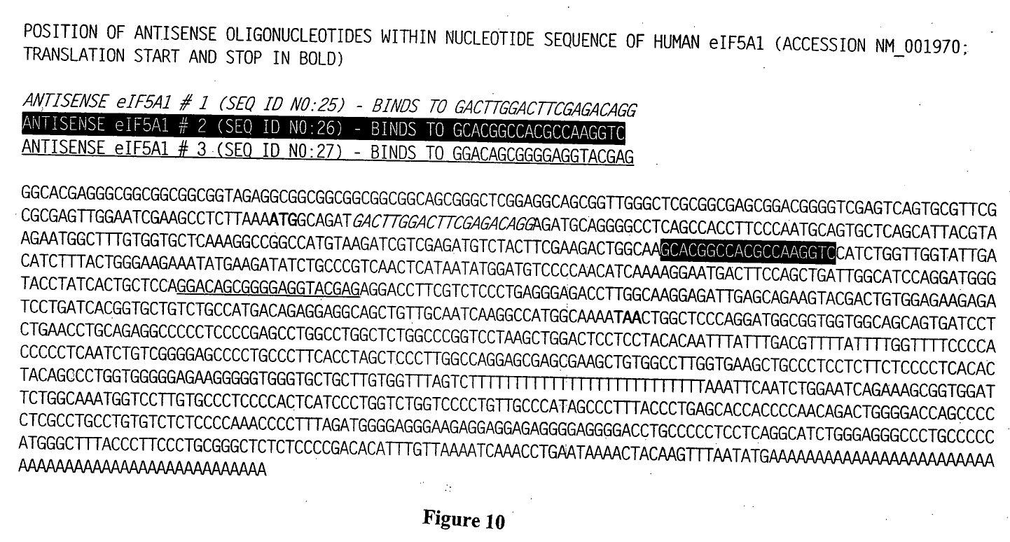

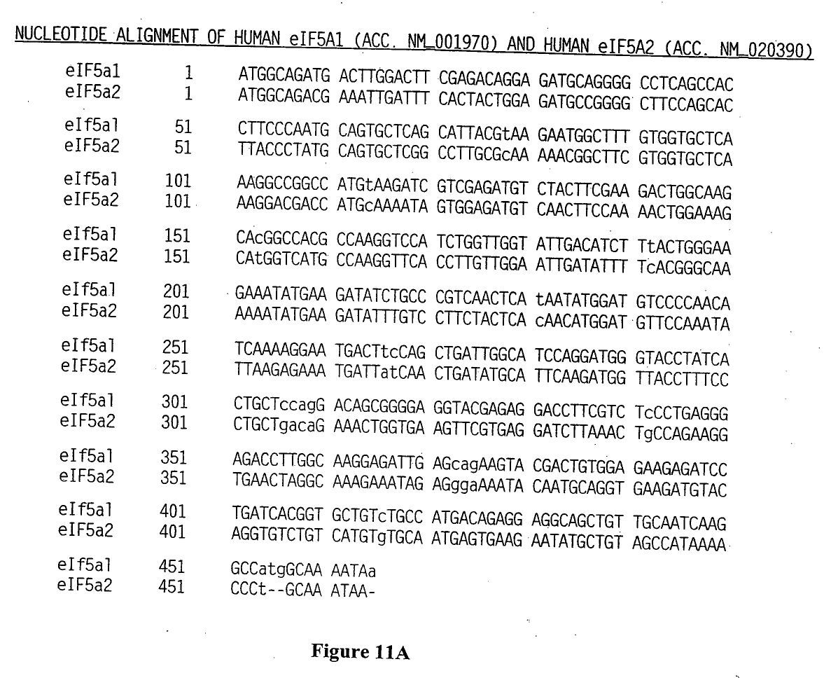

Exemplary antisense polynucleotides are shown in FIGS. 9-10. Any antisense eIF-5A1 polynucleotide or eIF-5A1 siRNA that inhibits expression of eIF-5A1 may be used in method or pharmaceutical compositions of the present invention.

The present invention also provides methods of inhibiting expression of p24 with siRNA or antisense polynucleotides of apoptosis specific eIF-5A. As discussed above, by inhibiting expression of eIF-5A1 with siRNA or antisense polynucleotides, expression of p24 is also inhibited.

Antisense oligonucleotides have been successfully used to accomplish both in vitro as well as in vivo gene-specific suppression. Antisense oligonucleotides are short, synthetic strands of DNA (or DNA analogs), RNA (or RNA analogs), or DNA/RNA hybrids that are antisense (or complimentary) to a specific DNA or RNA target. Antisense oligonucleotides are designed to block expression of the protein encoded by the DNA or RNA target by binding to the target mRNA and halting expression at the level of transcription, translation, or splicing. By using modified backbones that resist degradation (Blake et al., 1985), such as replacement of the phosphodiester bonds in the oligonucleotides with phosphorothioate linkages to retard nuclease degradation (Matzura and Eckstein, 1968), antisense oligonucleotides have been used successfully both in cell cultures and animal models of disease (Hogrefe, 1999). Other modifications to the antisense oligonucleotide to render the oligonucleotide more stable and resistant to degradation are known and understood by one skilled in the art. Antisense oligonucleotide as used herein encompasses double stranded or single stranded DNA, double stranded or single stranded RNA, DNA/RNA hybrids, DNA and RNA analogs, and oligonucleotides having base, sugar, or backbone modifications. The oligonucleotides may be modified by methods known in the art to increase stability, increase resistance to nuclease degradation or the like. These modifications are known in the art and include, but are not limited to modifying the backbone of the oligonucleotide, modifying the sugar moieties, or modifying the base.

Preferably, the antisense oligonucleotides of the present invention have a nucleotide sequence encoding a portion or the entire coding sequence of an eIF-5A1 polypeptide. In certain embodiments, the antisense oligonucleotides recognize the 5′end of the eIF-5A1.

The present invention contemplates the use of many suitable nucleic acid sequences encoding an eIF-5A1 polypeptide. For example, the present invention provides antisense oligonucleotides of the following eIF-5A1 nucleic acid sequences (SEQ ID NOS:1, 3, 4, 5, 11, 12, 15, 16, 19, 20, and 21) as well as other antisense nucleotides described herein. Antisense oligonucleotides of the present invention need not be the entire length of the provided SEQ ID NOs. They need only be long enough to be able to bind to inhibit or reduce expression of eIF-5A1. “Inhibition or reduction of expression” or “suppression of expression” refers to the absence or detectable decrease in the level of protein and/or mRNA product from a target gene, such as eIF-5A1.

Exemplary antisense oligonucleotides of eIF-5A1 that do not comprise the entire coding sequence are antisense oligonucleotides of eIF-5A1 having the following SEQ ID NO: 63, 64, 65, 25, 26, and 27.

“Antisense oligonucleotide of eIF-5A1” includes oligonucleotides having substantial sequence identity or substantial homology to eIF-5A1. Additional antisense oligonucleotides of eIF-5A1 of the present invention include those that have substantial sequence identity to those enumerated above (i.e. 90% homology) or those having sequences that hybridize under highly stringent conditions to the enumerated SEQ ID NOs. As used herein, the term “substantial sequence identity” or “substantial homology” is used to indicate that a sequence exhibits substantial structural or functional equivalence with another sequence. Any structural or functional differences between sequences having substantial sequence identity or substantial homology will be de minimus; that is, they will not affect the ability of the sequence to function as indicated in the desired application. Differences may be due to inherent variations in codon usage among different species, for example. Structural differences are considered de minimus if there is a significant amount of sequence overlap or similarity between two or more different sequences or if the different sequences exhibit similar physical characteristics even if the sequences differ in length or structure. Such characteristics include, for example, the ability to hybridize under defined conditions, or in the case of proteins, immunological crossreactivity, similar enzymatic activity, etc. The skilled practitioner can readily determine each of these characteristics by art known methods.

Additionally, two nucleotide sequences are “substantially complementary” if the sequences have at least about 70 percent or greater, more preferably 80 percent or greater, even more preferably about 90 percent or greater, and most preferably about 95 percent or greater sequence similarity between them. Two amino acid sequences are substantially homologous if they have at least 70%, more preferably at least 80%, even more preferably at least 90%, and most preferably at least 95% similarity between the active, or functionally relevant, portions of the polypeptides.

To determine the percent identity of two sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, at least 30%, 40%, 50%, 60%, 70%, 80%, or 90% or more of the length of a reference sequence is aligned for comparison purposes. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

The nucleic acid and protein sequences of the present invention can further be used as a “query sequence” to perform a search against sequence databases to, for example, identify other family members or related sequences. Such searches can be performed using the NBLAST and XBLAST programs (version 2.0) of Altschul, et al. (1990) J. Mol Biol. 215:403-10. BLAST nucleotide searches can be performed with the NBLAST program. BLAST protein searches can be performed with the XBLAST program to obtain amino acid sequences homologous to the proteins of the invention. To obtain gapped alignments for comparison purposes, Gapped BLAST can be utilized as described in Altschul et al. (1997) Nucleic Acids Res. 25(17):3389-3402. When utilizing BLAST and gapped BLAST programs, the default parameters of the respective programs (e.g., XBLAST and NBLAST) can be used.

The term “eIF-5A1” includes functional derivatives thereof. The term “functional derivative” of a nucleic acid is used herein to mean a homolog or analog of the amino acid or nucleotide sequence. A functional derivative retains the function of the given gene, which permits its utility in accordance with the invention. “Functional derivatives” of the eIF-5A1 polypeptide or functional derivatives of antisense oligonucleotides of eIF-5A1 as described herein are fragments, variants, analogs, or chemical derivatives of eIF-5A1 that retain eIF-5A1 activity or immunological cross reactivity with an antibody specific for eIF-5A1. A fragment of the eIF-5A1 polypeptide refers to any subset of the molecule.

Functional variants can also contain substitutions of similar amino acids that result in no change or an insignificant change in function. Amino acids that are essential for function can be identified by methods known in the art, such as site-directed mutagenesis or alanine-scanning mutagenesis (Cunningham et al. (1989) Science 244:1081-1085). The latter procedure introduces single alanine mutations at every residue in the molecule. The resulting mutant molecules are then tested for biological activity such as kinase activity or in assays such as an in vitro proliferative activity. Sites that are critical for binding partner/substrate binding can also be determined by structural analysis such as crystallization, nuclear magnetic resonance or photoaffinity labeling (Smith et al. (1992) J. Mol. Biol. 224:899-904; de Vos et al. (1992) Science 255:306-312).

A “variant” refers to a molecule substantially similar to either the entire gene or a fragment thereof, such as a nucleotide substitution variant having one or more substituted nucleotides, but which maintains the ability to hybridize with the particular gene or to encode mRNA transcript which hybridizes with the native DNA. A “homolog” refers to a fragment or variant sequence from a different animal genus or species. An “analog” refers to a non-natural molecule substantially similar to or functioning in relation to the entire molecule, a variant or a fragment thereof.

Variant peptides include naturally occurring variants as well as those manufactured by methods well known in the art. Such variants can readily be identified/made using molecular techniques and the sequence information disclosed herein. Further, such variants can readily be distinguished from other proteins based on sequence and/or structural homology to the eIF-5A1 of the present invention. The degree of homology/identity present will be based primarily on whether the protein is a functional variant or non-functional variant, the amount of divergence present in the paralog family and the evolutionary distance between the orthologs.

Non-naturally occurring variants of the eIF-5A1 polynucleotides, antisense oligonucleotides, or proteins of the present invention can readily be generated using recombinant techniques. Such variants include, but are not limited to deletions, additions and substitutions in the nucleotide or amino acid sequence. For example, one class of substitutions are conserved amino acid substitutions. Such substitutions are those that substitute a given amino acid in a protein by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe and Tyr. Guidance concerning which amino acid changes are likely to be phenotypically silent are found in Bowie et al., Science 247:1306-1310 (1990).

The term “hybridization” as used herein is generally used to mean hybridization of nucleic acids at appropriate conditions of stringency as would be readily evident to those skilled in the art depending upon the nature of the probe sequence and target sequences. Conditions of hybridization and washing are well known in the art, and the adjustment of conditions depending upon the desired stringency by varying incubation time, temperature and/or ionic strength of the solution are readily accomplished. See, e.g. Sambrook, J. et al., Molecular Cloning: A Laboratory Manual, 2^{nd} edition, Cold Spring Harbour Press, Cold Spring Harbor, N.Y., 1989.

The choice of conditions is dictated by the length of the sequences being hybridized, in particular, the length of the probe sequence, the relative G-C content of the nucleic acids and the amount of mismatches to be permitted. Low stringency conditions are preferred when partial hybridization between strands that have lesser degrees of complementarity is desired. When perfect or near perfect complementarity is desired, high stringency conditions are preferred. High stringency conditions means that the hybridization solution contains 6×S.S.C., 0.01 M EDTA, 1× Denhardt's solution and 0.5% SDS. Hybridization is carried out at about 68° C. for about 3 to 4 hours for fragments of cloned DNA and for about 12 to 16 hours for total eucaryotic DNA. For lower stringencies, the temperature of hybridization is reduced to about 42° C. below the melting temperature (Tₘ) of the duplex. The Tₘ is known to be a function of the G-C content and duplex length as well as the ionic strength of the solution.

As used herein, the phrase “hybridizes to a corresponding portion” of a DNA or RNA molecule means that the molecule that hybridizes, e.g., oligonucleotide, polynucleotide, or any nucleotide sequence (in sense or antisense orientation) recognizes and hybridizes to a sequence in another nucleic acid molecule that is of approximately the same size and has enough sequence similarity thereto to effect hybridization under appropriate conditions. For example, a 100 nucleotide long sense molecule will recognize and hybridize to an approximately 100 nucleotide portion of a nucleotide sequence, so long as there is about 70% or more sequence similarity between the two sequences. It is to be understood that the size of the “corresponding portion” will allow for some mismatches in hybridization such that the “corresponding portion” may be smaller or larger than the molecule which hybridizes to it, for example 20-30% larger or smaller, preferably no more than about 12-15% larger or smaller.

The present invention also provides other agents that can inhibit or reduce expression of eIF-5A1. One such agent includes small inhibitory RNAs (“siRNA”). Exemplary eIF5A1 siRNAs include SEQ ID NOs: 30, 31, 32, 33, dTdTCGACCUGAGGAGGAUGUGU or dTdTUCCUUACUGAAGGUCGACU

siRNA technology has been emerging as a viable alternative to antisense oligonucleotides since lower concentrations are required to achieve levels of suppression that are equivalent or superior to those achieved with antisense oligonucleotides (Thompson, 2002). Long double-stranded RNAs have been used to silence the expression of specific genes in a variety of organisms such as plants, nematodes, and fruit flies. An RNase-III family enzyme called Dicer processes these long double stranded RNAs into 21-23 nucleotide small interfering RNAs which are then incorporated into an RNA-induced silencing complex (RISC). Unwinding of the siRNA activates RISC and allows the single-stranded siRNA to guide the complex to the endogenous mRNA by base pairing. Recognition of the endogenous mRNA by RISC results in its cleavage and consequently makes it unavailable for translation. Introduction of long double stranded RNA into mammalian cells results in a potent antiviral response, which can be bypassed by use of siRNAs. (Elbashir et al., 2001). siRNA has been widely used in cell cultures and routinely achieves a reduction in specific gene expression of 90% or more.

The use of siRNAs has also been gaining popularity in inhibiting gene expression in animal models of disease. A recent study demonstrated that an siRNA against luciferase was able to block luciferase expression from a co-transfected plasmid in a wide variety of organs in post-natal mice. (Lewis et al., 2002). An siRNA against Fas, a receptor in the TNF family, injected hydrodynamically into the tail vein of mice was able to transfect greater than 80% of hepatocytes and decrease Fas expression in the liver by 90% for up to 10 days after the last injection (Song et al., 2003). The Fas siRNA was also able to protect mice from liver fibrosis and fulminant hepatitis. The development of sepsis in mice treated with a lethal dose of lipopolysaccharide was inhibited by the use of an siRNA directed against TNF-α (Sørensen et al., 2003). SiRNA has the potential to be a very potent drug for the inhibition of specific gene expression in vitro in light of their long-lasting effectiveness in cell cultures and their ability to transfect cells in vivo and their resistance to degradation in serum in vivo (Bertrand et al., 2002) in vivo.

Additional siRNAs include those that have substantial sequence identity to those enumerated (i.e. 90% homology) or those having sequences that hybridize under highly stringent conditions to the enumerated SEQ ID NOs. What is meant by substantial sequence identity and homology is described above with respect to antisense oligonucleotides of the present invention. The term “siRNAs of eIF-5A1” include functional variants or derivatives as described above with respect to antisense oligonucleotides of the present invention.

Delivery of siRNA and expression constructs/vectors comprising siRNA are known by those skilled in the art. U.S. applications 2004/106567 and 2004/0086884, which are herein incorporated by reference in their entirety, provide numerous expression constructs/vectors as well as delivery mechanism including viral vectors, non viral vectors, liposomal delivery vehicles, plasmid injection systems, artificial viral envelopes and poly-lysine conjugates to name a few.

One skilled in the art would understand regulatory sequences useful in expression constructs/vectors with antisense oligonucleotides or siRNA. For example, regulatory sequences may be a constitutive promoter, an inducible promoter, a tissue-specific promoter, or a combination thereof.

It is understood that the antisense nucleic acid and siRNAs of the present invention, where used in an animal for the purpose of prophylaxis or treatment, will be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers can further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The compositions of this invention can be in a variety of forms. These include, for example, solid, semi-solid and liquid dosage forms, such as tablets, pills, powders, liquid solutions, dispersions or suspensions, liposomes, suppositories, injectable and infusible solutions. The preferred form depends on the intended mode of administration and therapeutic application.

Such compositions can be prepared in a manner well known in the pharmaceutical art. In making the composition the active ingredient will usually be mixed with a carrier, or diluted by a carrier, and/or enclosed within a carrier which can, for example, be in the form of a capsule, sachet, paper or other container. When the carrier serves as a diluent, it can be a solid, semi-solid, or liquid material, which acts as a vehicle, excipient or medium for the active ingredient. Thus, the composition can be in the form of tablets, lozenges, sachets, cachets, elixirs, suspensions, aerosols (as a solid or in a liquid medium), ointments containing for example up to 10% by weight of the active compound, soft and hard gelatin capsules, suppositories, injection solutions, suspensions, sterile packaged powders and as a topical patch.

Having now generally described the invention, the same will be more readily understood through reference to the following examples, which are provided by way of illustration. The Examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Detailed descriptions of conventional methods, such as those employed in the construction of vectors and plasmids, the insertion of nucleic acids encoding polypeptides into such vectors and plasmids, the introduction of plasmids into host cells, and the expression and determination thereof of genes and gene products can be obtained from numerous publication, including Sambrook, J. et al., (1989) Molecular Cloning: A Laboratory Manual, 2^{nd} ed., Cold Spring Harbor Laboratory Press. All references mentioned herein are incorporated in their entirety.

### EXAMPLES

### Example 1

### Eukaryotic Initiation Factor 5A (eIF-5A1) Inhibits p24 Antigen Production in Both Unstimulated and Stimulated U1 Cells

U1 cells are a subclone of U937 human monocytic cells chronically infected with HIV-1. U1 cells were transfected using the Amaxa Nucleofector Device (Amaxa kit V), with either siRNA specific for eIF-5A1, or a scrambled control siRNA sequence (hcon). The eIF-5A1 siRNA (named h5A1), which targets a region of the 3′UTR of the human eIF-5A1 mRNA (Accession No. NM_{—}001970), had the following sequence: sense strand, 5′-GCUGGACUCCUCCUACACAdTdT-3′; antisense strand, 3′-dTdTCGACCUGAGGAGGAUGUGU-5′.

A second siRNA directed against eIF-5A, which targets an area in the coding region of the human eIF-5A mRNA (Accession No. NM_{—}001970) had the following sequence: sense strand, 5′-AGGAAUGACUUCCAGCUGAdTdT-3′; antisense strand, 3′-dTdTUCCUUACUGAAGGUCGACU-5′.

The control siRNA that was used had the reverse sequence of the eIF-5A1 specific siRNA (h5A1) and had no identity to any known human gene product. The control siRNA had the following sequence: sense strand, 5′-ACACAUCCUCCUCAGGUCGdTdT-3′; antisense strand, 3′-dTdTUGUGUAGGAGGAGUCCAGC-5′.

24 hours after electroporation, the cells were counted, resuspended in fresh medium and plated at 1×10⁶ cells/ml in 0.5 ml volume in 24-well tissue culture plates. The cells were incubated for an additional 24 hours with either medium alone, or medium containing IL-18 as a stimulus. After 24 hours, Triton-X-100 (1% v/v) was added to each well and samples were assayed for p24 HIV-1 antigen by ELISA. Results were calculated as percent p24 production compared to Control (electroporated cells in the absence of siRNA). Results are graphed for cells cultured in medium alone (open bars), or for cells stimulated with IL-18 (closed bars). As shown in FIG. 16, 7 μg eIF-5A1 inhibited p24 production by 42% and 51% when added to cells in medium or cells exposed to IL-18, respectively. Shown are data from 4-6 separate experiments.

### Example 2

### eIF-5A1 siRNA Inhibits Interleukin 8 (IL-8) Production in both Unstimulated and Stimulated U1 Cells

FIG. 17 shows IL-8 concentrations measured in samples. Percent IL-8 production was calculated as for p24. As shown in FIG. 17, 7 μg eIF-5A1 siRNA (the siRNAs are as described in Example 1) inhibited IL-8 production by 41% and 47% when added to cells in medium or cells exposed to IL-18, respectively. The data are represented as percent IL-8 production compared to Control (electroporated cells in the absence of siRNA). Shown are data from 4-5 separate experiments.

1. A method for inhibiting replication of HIV virus comprising inhibiting expression of eIF-5A1 with an agent capable of inhibiting expression of eIF-5A1 wherein said inhibition of expression of eIF-5A1 inhibits expression of p24 thereby inhibiting HIV replication.

2. The method of claim 1 wherein the agent is an antisense polynucleotide of eIF-5A1 or is an siRNA targeted at eIF-5A1.

3. The method of claim 2 wherein the antisense polynucleotide is selected from the group of antisense polynucleotides consisting of SEQ ID NO: 63, SEQ ID NO: 64, SEQ ID NO: 65; SEQ ID NO:25, SEQ ID NO:26, and SEQ ID NO:27.

4. The method of claim 2 wherein the siRNA targeted at eIF-5A1 is selected from the group of siRNAs consisting of SEQ ID NO: 46, SEQ ID NO: 48, SEQ ID NO: 51, and SEQ ID NO: 54.

5. The method of claim 2 wherein the siRNA has the following sequence: dTdTCGACCUGAGGAGGAUGUGU (SEQ ID NO: 96) or dTdTUCCUUACUGAAGGUCGACU (SEQ ID NO: 97).

6. The method of claim 1 wherein the method comprises transfecting a cell infected with HIV with an siRNA or antisense polynucleotide of eIF-5A1.

7. A method of inhibiting expression of p24 in a HIV infected cell, comprising inhibiting expression of eIF-5A1 with an agent capable of inhibiting expression of eIF-5A1 wherein said inhibition of expression of eIF-5A1 inhibits expression of p24.

8. The method of claim 7 wherein the agent is an antisense polynucleotide of eIF-5A1 or is an siRNA targeted at eIF-5A1.

9. The method of claim 8 wherein the antisense polynucleotide is selected from the group of antisense polynucleotides consisting of SEQ ID NO: 63, SEQ ID NO: 64, SEQ ID NO: 65; SEQ ID NO:25, SEQ ID NO:26, and SEQ ID NO:27.

10. The method of claim 8 wherein the siRNA targeted at eIF-5A1 is selected from the group of siRNAs consisting of SEQ ID NO: 46, SEQ ID NO: 48, SEQ ID NO: 51, and SEQ ID NO: 54.

11. The method of claim 8 wherein the siRNA has the following sequence: dTdTCGACCUGAGGAGGAUGUGU (SEQ ID NO: 96) or dTdTUCCUUACUGAAGGUCGACU (SEQ ID NO: 97).

12. The method of claim 7 wherein the method comprises transfecting a cell infected with HIV with an siRNA or antisense polynucleotide of eIF-5A1.

13. A pharmaceutical composition for inhibiting HIV replication or inhibiting p24 expression in an HIV infected cell comprising an antisense polynucleotide selected from the group of antisense polynucleotides consisting of SEQ ID NO: 63, SEQ ID NO: 64, SEQ ID NO: 65; SEQ ID NO:25, SEQ ID NO:26, and SEQ ID NO:27.

14. A pharmaceutical composition for inhibiting HIV replication or inhibiting p24 expression in an HIV infected cell comprising siRNA targeted at eIF-5A1 selected from the group of siRNAs consisting of SEQ ID NO: 46, SEQ ID NO: 48, SEQ ID NO: 51, and SEQ ID NO: 54.

15. A pharmaceutical composition for inhibiting HIV replication or inhibiting p24 expression in an HIV infected cell comprising eIF-5A1 siRNA having the following sequence: dTdTCGACCUGAGGAGGAUGUGU (SEQ ID NO: 96) or dTdTUCCUUACUGAAGGUCGACU (SEQ ID NO: 97).
